SEQUENCE LISTING (18 sequences)

SEQ ID NO: 1 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatg

SEQ ID NO: 2 - protein, 189 aa, Homo sapiens [sequence omitted]

SEQ ID NO: 3 - DNA, 567 bp, Homo sapiens
atgactgaatataaacttgtggtagttggagcttgtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatg

SEQ ID NO: 4 - protein, 189 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 5 - DNA, 567 bp, Homo sapiens
atgactgaatataaacttgtggtagttggagctgatggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatg

SEQ ID NO: 6 - protein, 189 aa, Homo sapiens
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 7 - DNA, 567 bp, Homo sapiens
atgactgaatataaacttgtggtagttggagctgttggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatg

SEQ ID NO: 8 - protein, 189 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 9 - DNA, 517 bp, Homo sapiens
atgactgaatataaacttgtggtagttggagctggtggcgtaggcaagagcaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 10 - protein, 188 aa, Homo sapiens [sequence omitted]

SEQ ID NO: 11 - DNA, 517 bp, Homo sapiens
atgactgaatataaacttgtggtagttggagcttgtggcgtaggcaagagcaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 12 - protein, 188 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 13 - DNA, 517 bp, Homo sapiens
atgactgaatataaacttgtggtagttggagctgatggcgtaggcaagagcaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 14 - protein, 188 aa, Homo sapiens [sequence omitted]

SEQ ID NO: 15 - DNA, 517 bp, Homo sapiens
atgactgaatataaacttgtggtagttggagctgttggcgtaggcaagagcaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 16 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 17 - DNA, 549 bp, Artificial sequence
  Synthetic NanoLuc
atgaaacatcaccatcaccatcatgcgatcgccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggtt

SEQ ID NO: 18 - protein, 183 aa, Artificial sequence
  Synthetic NanoLuc
MKHHHHHHAIAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAV

(19) United States
(12) Patent Application Publication
(10) Pub. No.: US 2021/0363565 A1
(43) Pub. Date: Nov. 25, 2021

(54) ENHANCEMENT OF KINASE TARGET ENGAGEMENT

(71) Applicant: Promega Corporation, Madison, WI (US)

(72) Inventors: James Vasta, Waunakee, WI (US); Matthew Robers, Fitchburg, WI (US)

(21) Appl. No.: 17/327,319

(22) Filed: May 21, 2021

Related U.S. Application Data
(60) Provisional application No. 63/109,103, filed on Nov. 3, 2020.
(60) Provisional application No. 63/028,729, filed on May 22, 2020.

(51) Int. Cl. C12Q 1/48; G01N 21/64; G01N 24/00

(52) U.S. Cl. CPC C12Q 1/485; G01N 21/64; G01N 2333/914; G01N 2021/6439; G01N 24/00

(57) ABSTRACT

Provided herein are systems and methods for enhanced engagement of protein kinases by kinase binding agents. In particular, the engagement of kinases by functional kinase binding agents is enhanced by the co-expression of the kinases with an active variant of KRAS.

### CROSS-REFERENCE TO RELATED APPLICATION

The present application claims priority to U.S. Provisional Patent Application Ser. No. 63/028,729, filed May 22, 2020, and U.S. Provisional Patent Application Ser. No. 63/109,103, filed Nov. 3, 2020, both of which are hereby incorporated by reference in their entireties.

### SEQUENCE LISTING

The text of the computer readable sequence listing filed herewith, titled “38511-203_SEQUENCE_LISTING_ST25”, created May 21, 2021, having a file size of 22,729 bytes, is hereby incorporated by reference in its entirety.

### FIELD

Provided herein are systems and methods for enhanced engagement of protein kinases by kinase binding agents. In particular, the engagement of kinases by functional kinase binding agents is enhanced by the co-expression of the kinases with an active variant of KRAS.

### BACKGROUND

The human genome contains about 560 protein kinase genes, and they constitute about 2% of all human genes (Manning et al. (2002) Science 298 (5600): 1912-1934; herein incorporated by reference in its entirety). Up to 30% of all human proteins may be modified by kinase activity, and kinases are known to regulate the majority of cellular pathways, especially those involved in signal transduction. The chemical activity of a kinase involves transferring a phosphate group from a nucleoside triphosphate (usually ATP) and covalently attaching it to specific amino acids with a free hydroxyl group. Most kinases act on both serine and threonine (serine/threonine kinases), others act on tyrosine (tyrosine kinases), and a number act on all three (dual-specificity kinases) (Dhanasekaran & Premkumar (September 1998). Oncogene. 17 (11 Reviews): 1447-55; herein incorporated by reference in its entirety). Aberrant kinase signaling is associated with many diseases and conditions.

Even with improved kinase ligands with broad specificity, some kinases are difficult to target and engage.

The KRAS gene encodes the KRAS protein, which is part of the RAS/MAPK pathway. KRAS relays signals from outside the cell to the cell's nucleus that instruct the cell to grow, divide, mature, and/or differentiate. KRAS is a GTPase that acts as a molecular switch, turning on and off by the conversion of GTP to GDP. The KRAS gene is an oncogene and, when mutated, can cause normal cells to become cancerous. KRAS-activating mutations are the most frequent oncogenic alterations in human cancer. One common KRAS-activating mutation that drives neoplastic transformation in cells is KRAS^{G12C}. KRAS-activating mutations such as KRAS^{G12C} fix the KRAS protein in its active GTP-bound form by interfering with the GTP to GDP cycling process.

### SUMMARY

Provided herein are systems and methods for enhanced engagement of protein kinases by kinase binding agents. In particular, the engagement of kinases by functional kinase binding agents is enhanced by the co-expression of the kinases with an active variant of KRAS.

In some embodiments, provided herein are methods of detecting or quantifying a kinase in a sample, comprising: (a) providing a sample comprising the kinase and an active KRAS variant; and (b) contacting the sample with a kinase binding agent comprising a kinase binding moiety. In some embodiments, the kinase binding agent is a functional kinase binding agent and comprises a kinase binding moiety and a functional element. In some embodiments, the kinase binding agent consists of a kinase binding moiety. In some embodiments, methods further comprise (c) detecting or quantifying the functional element. In some embodiments, step (a) comprises contacting a sample comprising the kinase with the active KRAS variant. In some embodiments, step (a) comprises expressing the kinase and the active KRAS variant within the sample. In some embodiments, the active KRAS variant is an active variant of the KRAS4A isoform (e.g., KRAS4A^{G12C}, KRAS4A^{G12D}, KRAS4A^{G12V}, etc.). In some embodiments, the active KRAS variant is an active variant of the KRAS4B isoform (e.g., KRAS4B^{G12C}, KRAS4B^{G12D}, KRAS4B^{G12V}, etc.). In some embodiments, the active KRAS variant is a KRAS^{G12C} variant (e.g., KRAS4B^{G12C}, KRAS4B^{G12C}, etc.) In some embodiments, the functional element is a detectable element, an affinity element, a capture element, or a solid support. In some embodiments, the functional element is a detectable element selected from a fluorophore, chromophore, radionuclide, electron opaque molecule, an MRI contrast agent, SPECT contrast agent, and mass tag. In some embodiments, the detectable element, or the signal produced thereby, is detected or quantified by fluorescence, mass spectrometry, optical imaging, magnetic resonance imaging (MRI), or energy transfer. In some embodiments, the functional element is a solid support selected from a sedimental particle, a membrane, glass, a tube, a well, a self-assembled monolayer, a surface plasmon resonance chip, and a solid support with an electron conducting surface. In some embodiments, the sedimental particle is a magnetic particle. In some embodiments, the broad-spectrum kinase binding agent is of the formula:

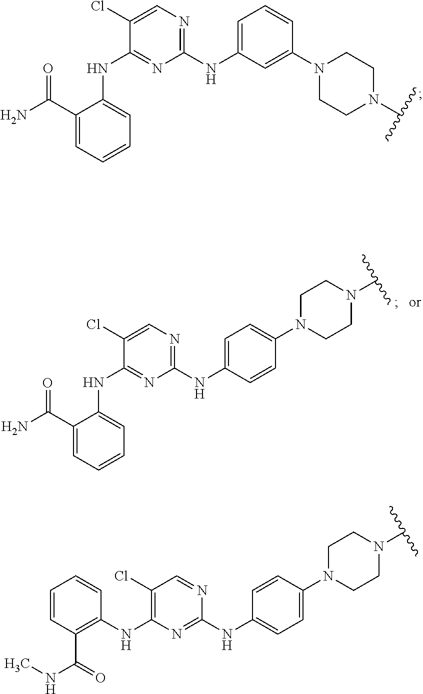 [SMILES: *N1CCN(c2cccc(Nc3ncc(Cl)c(Nc4ccccc4C(N)=O)n3)c2)CC1 ; *N1CCN(c2ccc(Nc3ncc(Cl)c(Nc4ccccc4C(N)=O)n3)cc2)CC1 ; *N1CCN(c2ccc(Nc3ncc(Cl)c(Nc4ccccc4C(=O)NC)n3)cc2)CC1]

and is attached to the detectable functional element. In some embodiments, the sample is selected from a cell, cell lysate, body fluid, tissue, biological sample, in vitro sample, and environmental sample. In some embodiments, the kinase is expressed as a fusion with a bioluminescent reporter. In some embodiments, the bioluminescent reporter is a luciferase with at least 70% sequence identity with SEQ ID NO: 4. In some embodiments, the emission spectrum of the bioluminescent reporter and the excitation spectrum of the functional element overlap. In some embodiments, methods further comprise contacting the sample with a substrate for the bioluminescent reporter. In some embodiments, the substrate is coelenterazine, a coelenterazine derivative, or furimazine.

In some embodiments, provided herein are systems comprising: (a) a target kinase (e.g., a plurality of target kinases); (b) an active variant of KRAS; and (c) a kinase binding agent comprising a kinase binding moiety. In some embodiments, the kinase binding agent is a functionalized kinase binding agent and comprises a kinase binding moiety and a functional element. In some embodiments, the kinase binding agent consists of a kinase binding moiety. In some embodiments, the system comprises a cell, cell lysate, tissue, or cell-free system. In some embodiments, the kinase and the active KRAS variant are expressed within the system. In some embodiments, the active KRAS variant is an active variant of the KRAS4A isoform (e.g., KRAS4A^{G12C}, KRAS4A^{G12D}, KRAS4A^{G12V}, etc.). In some embodiments, the active KRAS variant is an active variant of the KRAS4B isoform (e.g., KRAS4B^{G12C}, KRAS4B^{G12D}, KRAS4B^{G12V}, etc.). In some embodiments, the active KRAS variant is a KRAS^{G12C} variant (e.g., KRAS4B^{G12C}, KRAS4B^{G12C}, etc.). In some embodiments, the functional element is a detectable element, an affinity element, a capture element, or a solid support. In some embodiments, the functional element is a detectable element selected from a fluorophore, chromophore, radionuclide, electron opaque molecule, an MRI contrast agent, SPECT contrast agent, and mass tag. In some embodiments, the detectable element, or the signal produced thereby, is detectable or quantifiable by fluorescence, mass spectrometry, optical imaging, magnetic resonance imaging (MRI), or energy transfer. In some embodiments, the functional element is a solid support selected from a sedimental particle, a membrane, glass, a tube, a well, a self-assembled monolayer, a surface plasmon resonance chip, and a solid support with an electron conducting surface. In some embodiments, the sedimental particle is a magnetic particle. In some embodiments, the kinase binding agent is general kinase inhibitor or a specific kinase inhibitor (e.g., a drug molecule that binds to and inhibits one or more kinases). In some embodiments, the broad-spectrum kinase binding agent is of the formula:

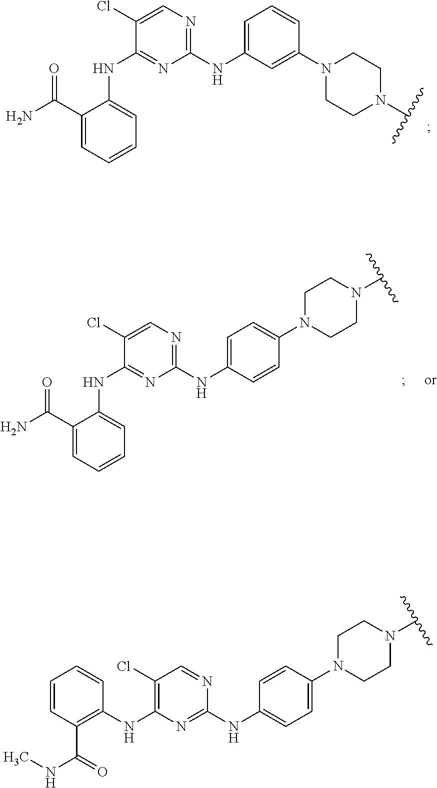 [SMILES: *N1CCN(c2cccc(Nc3ncc(Cl)c(Nc4ccccc4C(N)=O)n3)c2)CC1 ; *N1CCN(c2ccc(Nc3ncc(Cl)c(Nc4ccccc4C(N)=O)n3)cc2)CC1 ; *N1CCN(c2ccc(Nc3ncc(Cl)c(Nc4ccccc4C(=O)NC)n3)cc2)CC1]

and is attached to the detectable functional element. In some embodiments, the system comprises a sample is selected from a cell, cell lysate, body fluid, tissue, biological sample, in vitro sample, and environmental sample. In some embodiments, the kinase is present as a fusion with a bioluminescent reporter. In some embodiments, the bioluminescent reporter is a luciferase with at least 70% sequence identity with SEQ ID NO: 4. In some embodiments, the emission spectrum of the bioluminescent reporter and the excitation spectrum of the functional element overlap. In some embodiments, systems further comprise a substrate for the bioluminescent reporter. In some embodiments, the substrate is coelenterazine, a coelenterazine derivative, or furimazine.

In some embodiments, the systems and methods provided herein utilize functional kinase binding agents which comprise a first moiety capable of bind to a kinase protein (e.g., a broad spectrum of kinase proteins) and second functional element (e.g., detectable element, capture element, affinity element, solid support, etc.), such as those described in U.S. Pub No. 2020/000771; incorporated by reference in its entirety.

In some embodiments, provided herein are functional kinase binding agents of formula:

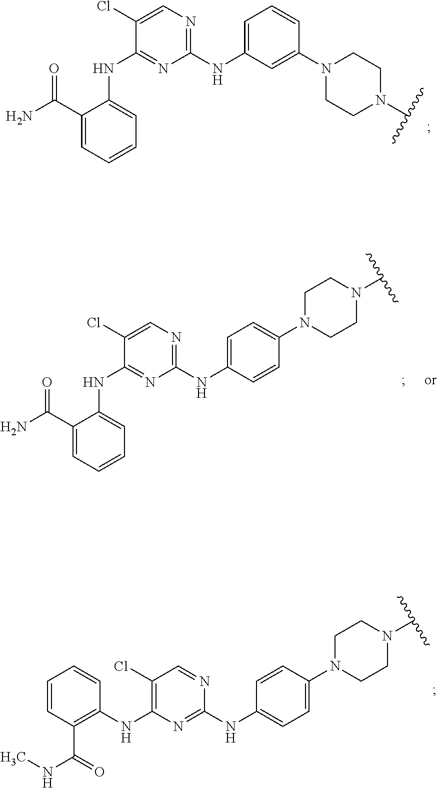 [SMILES: *N1CCN(c2cccc(Nc3ncc(Cl)c(Nc4ccccc4C(N)=O)n3)c2)CC1 ; *N1CCN(c2ccc(Nc3ncc(Cl)c(Nc4ccccc4C(N)=O)n3)cc2)CC1 ; *N1CCN(c2ccc(Nc3ncc(Cl)c(Nc4ccccc4C(=O)NC)n3)cc2)CC1]

wherein the kinase binding moieties above are linked to a function element (e.g., detectable element, capture element, affinity element, solid surface, etc.). In some embodiments, a detectable element comprises a fluorophore, chromophore, radionuclide, electron opaque molecule, an MRI contrast agent, SPECT contrast agent, or mass tag. In some embodiments, a solid surface is selected from a sedimental particle, a membrane, glass, a tube, a well, a self-assembled monolayer, a surface plasmon resonance chip, or a solid support with an electron conducting surface. In some embodiments, the sedimental particle is a magnetic particle.

In some embodiments, a broad-spectrum kinase binding agent is attached to the detectable element directly. In some embodiments, a broad-spectrum kinase binding agent is attached to the detectable element via a linker. In some embodiments, the linker comprises —[(CH₂)₂O]ₙ—, wherein n is 1-20 (e.g., 1, 2, 3, 4, 5, 6, 7, 8, 9 10, 11, 12, 13, 14, 15, 16, 17, 18, 19 20, or ranges therebetween). In some embodiments, the linker is attached to the broad-spectrum kinase binding agent and/or the detectable element by an amide bond.

In some embodiments, provided herein are functional kinase binding agents comprising a structure of:

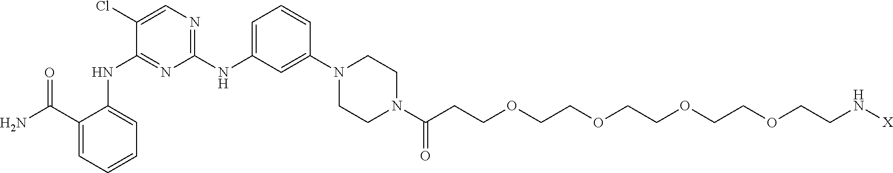

wherein X is a functional element (e.g., detectable element, capture element, affinity element, solid surface, etc.). In some embodiments, X is a fluorophore. In some embodiments, provided herein is a functional kinase binding agent comprising a structure of:

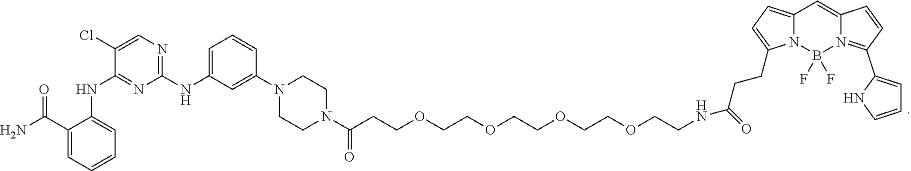

In some embodiments, provided herein is a functional kinase binding agent comprising a structure of:

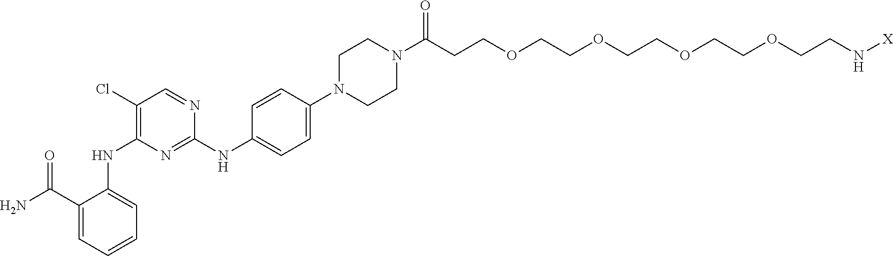

wherein X is a functional element (e.g., detectable element, capture element, affinity element, solid surface, etc.). In some embodiments, X is a fluorophore. In some embodiments, provided herein is a functional kinase binding agent comprising a structure of:

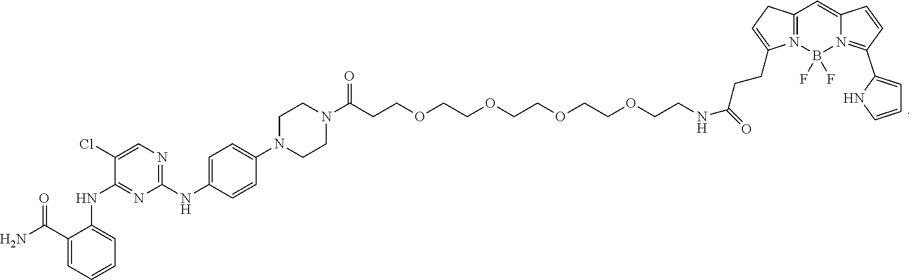

In some embodiments, provided herein is a functional kinase binding agent comprising a structure of:

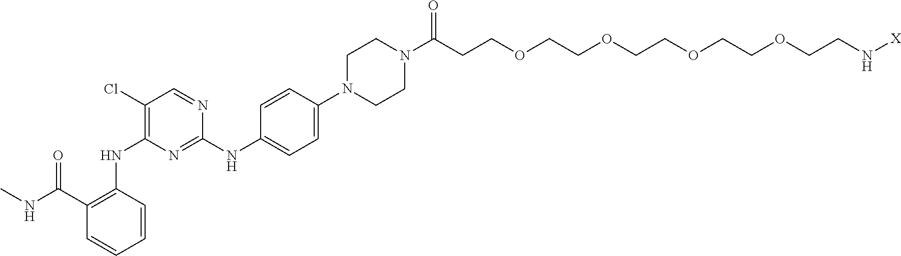

wherein X is a functional element (e.g., detectable element, capture element, affinity element, solid surface, etc.). In some embodiments, X is a fluorophore. In some embodiments, provided herein is a functional kinase binding agent comprising a structure of:

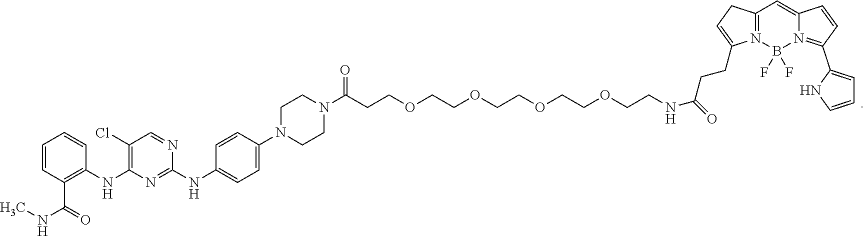

In some embodiments, a functional kinase binding agent comprises a non-natural abundance of one or more stable heavy isotopes.

In some embodiments, provided herein are methods of detecting or quantifying kinases in a sample comprising contacting the sample with a functional kinase binding agent and detecting or quantifying the detectable element or a signal produced thereby. In some embodiments, the detectable element, or a signal produced thereby, is detected or quantified by fluorescence, mass spectrometry, optical imaging, magnetic resonance imaging (MRI), or energy transfer (e.g., FRET, BRET, ALPHA).

In some embodiments, provided herein are methods of isolating kinases from a sample comprising contacting the sample with a functional kinase binding agent and separating the complex of the functional kinase binding agent and a bound kinase from the unbound portion of the sample based on the functionality of the functional element (e.g., capture element, affinity element, solid surface, etc.). In some embodiments, methods comprise isolating the kinases from a sample by a method described herein and analyzing the isolated kinases by mass spectrometry.

In some embodiments, provided herein are methods of monitoring interactions between kinases and unmodified biomolecules comprising contacting the sample with a functional kinase binding agent herein.

In some embodiments, methods herein are performed using a sample selected from a cell, cell lysate, body fluid, tissue, biological sample, in vitro sample, and environmental sample.

### BRIEF DESCRIPTION OF THE DRAWINGS

FIG. 1A-F. Impact of KRAS4B^{G12C} on kinase engagement. (A) Molecular structures of pan-kinase inhibitor CC1 and functional kinase binding agent K10. (B) Affinity of K10 tracer vs. CC1 standard for BRAF. (C) Affinity of K10 tracer vs. CC1 standard for JAK2. (D) Affinity of K10 tracer vs. CC1 standard for MAPK1. (E) Affinity of K10 tracer vs. CC1 standard for BTK. (F) Affinity of K10 tracer vs. CC1 standard for MAPK3.

FIG. 2. Enhanced BRAF kinase engagement with multiple kinase binding moieties in the presence of KRAS4B^{G12C}.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols as herein described as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an”, and “the” include plural reference unless the context clearly dictates otherwise. Thus, for example, reference to “a peptide” is a reference to one or more peptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “tracer” refers to a compound of interest or an agent that binds to an analyte of interest (e.g., protein of interest (e.g., kinase), etc.) and displays a moiety with a quantifiable or detectable property (e.g., detected or quantified any suitable biochemical or biophysical technique (e.g., optically, magnetically, electrically, by resonance imaging, by mass, by radiation, etc.)). Tracers may comprise a compound of interest or an agent that binds to an analyte of interest linked (e.g., directly or via a suitable linker) to a fluorophore, radionuclide, mass tag, contrast agent for magnetic resonance imaging (MRI), planar scintigraphy (PS), positron emission tomography (PET), single photon emission computed tomography (SPECT), and computed tomography (CT) (e.g., a metal ion chelator with bound metal ion, isotope, or radionuclide), etc.

As used herein, the term “sample” is used in its broadest sense. In one sense, it is meant to include a specimen or culture obtained from any source as well as biological and environmental samples. Biological samples may be obtained from animals (including humans) and encompass fluids, solids, tissues, and gases. Biological samples include blood products such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not however to be construed as limiting the sample types applicable to the present invention.

As used herein, the term “linearly connected atoms” refers to the backbone atoms of a chain or polymer, excluding pendant, side chain, or H atoms that do not form the main chain or backbone.

As used herein, the term “detectable element” refers to a detectable, reactive, affinity, or otherwise bioactive agent or moiety that is attached (e.g., directly or via a suitable linker) to a compound described herein derivatives or analogs thereof, etc.). Other additional detectable elements that may find use in embodiments described herein comprise “localization elements”, “detection elements”, etc.

As used herein, the term “capture element” refers to a molecular entity that forms a covalent interaction with a corresponding “capture agent.”

As used herein, the term “affinity element” refers to a molecular entity that forms a stable noncovalent interaction with a corresponding “affinity agent.”

As used herein, the term “solid support” is used in reference to any solid or stationary material to which reagents such as substrates, mutant proteins, drug-like molecules, and other test components are or may be attached. Examples of solid supports include microscope slides, wells of microtiter plates, coverslips, beads, particles, resin, cell culture flasks, as well as many other suitable items. The beads, particles, or resin can be magnetic or paramagnetic.

As used herein, in chemical structures the indication:

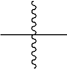

represents a point of attachment of one moiety to another moiety (e.g., kinase binding agent to a functional element).

“Coelenterazine” as used herein refers to naturally-occurring (“native”) coelenterazine. As used herein, the term “coelenterazine analog” or “coelenterazine derivative” refers to synthetic (e.g., derivative or variant) and natural analogs thereof, including furimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); U.S. Pat. No. 8,669,103; WO 2012/061529, U.S. Pat. Pub. 2017/0233789 and U.S. Pat. Pub. 2018/0030059; the disclosures of which are incorporated by reference herein in their entireties. In some embodiments, coelenterazine analogs include pro-substrates such as, for example, those described in U.S. application Ser. No. 12/056,073; U.S. Pub. No. 2012/0707849; U.S. Pub. No. 2014/0099654; herein incorporated by reference in their entireties.

“Peptide” and “polypeptide” as used herein, and unless otherwise specified, refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

“Variant” is used herein to describe a peptide or polypeptide that differs in amino acid sequence by the insertion, deletion, or conservative substitution of amino acids, but retain at least one biological activity. “SNP” refers to a variant that is a single nucleotide polymorphism. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid (e.g., replacing an amino acid with a different amino acid of similar properties, such as hydrophilicity, degree, and distribution of charged regions) is recognized in the art as typically involving a minor change. These minor changes can be identified, in part, by considering the hydropathic index of amino acids, as understood in the art. The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### DETAILED DESCRIPTION

Provided herein are systems and methods for enhanced engagement of protein kinases by kinase binding agents. In particular, the engagement of kinases by functional kinase binding agents is enhanced by the co-expression of the kinases with an active variant of KRAS.

In some embodiments, provided herein are systems and methods for the engagement of kinases in which an active variant of KRAS (e.g., KRAS4A variants, KRAS4B variants, variants of KRAS^{G12C}, variants of KRAS^{G12D}, variants of KRAS^{G12V}, etc.) is provided along with a functional kinase binding agent. The presence of the active KRAS protein (e.g., constitutively active) activates the RAS/MAPK pathway, and other kinase rich pathways associated therewith, thereby enhancing target engagement by functional kinase binding agent; however, embodiments herein are not limited to this mechanism of action and an understanding of the mechanism underlying the systems and methods herein is not necessary to practice the invention. The enhanced target engagement that occurs in the presence of an active variant of KRAS (e.g., KRAS4A variants, KRAS4B variants, variants of KRAS^{G12C}, variants of KRAS^{G12D}, variants of KRAS^{G12V}, etc.) provides systems and methods with enhanced detection, quantification, purification, isolation, etc. of kinases.

Although embodiments herein are described as being suitable for the detection/isolation of protein kinases, any embodiments herein may also find use in the detection/isolation of other proteins, for example, if the activity and/or expression of those proteins is enhanced by the presence/co-expression of the active KRAS (e.g., KRAS4A^{G12C}, KRAS4A^{G12D}, KRAS4A^{G12V}, KRAS4B^{G12C}, KRAS4B^{G12D}, KRAS4B^{G12V} etc.). For example, proteins that are activated/expressed in KRAS pathways (e.g., kinases, non-kinases), or pathways associated therewith, are more readily detected/isolated in the presence of an active variant of KRAS.

### I. Active KRAS Variants

In some embodiments, the active KRAS variant is an active variant of the KRAS4A isoform (e.g., KRAS4A^{G12C}, KRAS4A^{G12D} KRAS4A^{G12V}, etc.). In some embodiments, the active KRAS variant is an active variant of the KRAS4B isoform (e.g., KRAS4B^{G12C}, KRAS4B^{G12D}, KRAS4B^{G12V}, etc.).

In some embodiments, provided herein are systems for enhanced target engagement comprising an active variant of KRAS4A (SEQ ID NO: 2). In some embodiments, active variants of KRAS4A are provided, for example, active variants (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 2. In some embodiments, an active KRAS4A variant is provided with one or more substitutions (e.g., 1, 2, 3, 4, 5, 6, 7, 8, 9, 10, 15, 20, 25, 30, 40, 50, 60, or ranges therebetween) relative to SEQ ID NO: 2. In some embodiments, an active KRAS4A variant comprises a substitution at position 12. In some embodiments, provided herein are methods for enhanced target engagement comprising in which active variants of KRAS4A (SEQ ID NO: 2) are provided and/or expressed.

In some embodiments, provided herein are systems for enhanced target engagement comprising a nucleic acid (e.g., variants of SEQ ID NO: 1) encoding an active variant of KRAS4A. In some embodiments, sequences encoding active variants of KRAS4A are provided, for example, sequences encoding active variants (e.g., constitutively active) comprising at least 70% (e.g., 70%, 75%, 80%, 85%, 90%, 95%, 96%, 97%, 98%, 99%, 100%, or ranges therebetween) sequence identity with the KRAS4A sequence SEQ ID NO: 1. In some embodiments, sequences comprising at least 70% (e.g., 70%, 75%, 80%, 85%, 90%, 95%, 96%, 97%, 98%, 99%, 100%, or ranges therebetween) sequence identity with SEQ ID NO: 1 are provided. In some embodiments, a KRAS4A variant nucleotide sequence is provided with one or more substitutions (e.g., 1, 2, 3, 4, 5, 6, 7, 8, 9, 10, 15, 20, 25, 30, 40, 50, 60, or ranges therebetween) relative to SEQ ID NO: 1. In some embodiments, a KRAS4A variant nucleotide sequence comprises a substitution at one or more of positions 34, 35 or 36 of SEQ ID NO: 1. In some embodiments, provided herein are methods of enhanced target engagement comprising providing a KRAS4A variant nucleotide sequence (e.g., a variant of SEQ ID NO: 1) that encodes and active KRAS4A variant.

In some embodiments, provided herein are systems for enhanced target engagement comprising an active variant of KRAS4A^{G12C} (SEQ ID NO: 4). In some embodiments, active variants of KRAS4A^{G12C} are provided, for example, active variants (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 4. In some embodiments, an active KRAS4A^{G12C} variant is provided with one or more substitutions (e.g., 1, 2, 3, 4, 5, 6, 7, 8, 9, 10, 15, 20, 25, 30, 40, 50, 60, or ranges therebetween) relative to SEQ ID NO: 4. In some embodiments, an active KRAS4A^{G12C} variant comprises a C at position 12. In some embodiments, provided herein are methods for enhanced target engagement comprising in which active variants of KRAS4A^{G12C} (SEQ ID NO: 4) are provided and/or expressed.

In some embodiments, provided herein are systems for enhanced target engagement comprising a nucleic acid (e.g., variants of SEQ ID NO: 3) encoding an active variant of KRAS4A^{G12C}. In some embodiments, sequences encoding active variants of KRAS4A^{G12C} are provided, for example, sequences encoding active variants (e.g., constitutively active) comprising at least 70% (e.g., 70%, 75%, 80%, 85%, 90%, 95%, 96%, 97%, 98%, 99%, 100%, or ranges therebetween) sequence identity with the KRAS4A^{G34T} sequence SEQ ID NO: 3. In some embodiments, sequences comprising at least 70% (e.g., 70%, 75%, 80%, 85%, 90%, 95%, 96%, 97%, 98%, 99%, 100%, or ranges therebetween) sequence identity with SEQ ID NO: 3 are provided. In some embodiments, a KRAS4A^{G34T} variant nucleotide sequence is provided with one or more substitutions (e.g., 1, 2, 3, 4, 5, 6, 7, 8, 9, 10, 15, 20, 25, 30, 40, 50, 60, or ranges therebetween) relative to SEQ ID NO: 3. In some embodiments, a KRAS4A^{G34T} variant nucleotide sequence comprises a T at position 34 of SEQ ID NO: 3. In some embodiments, provided herein are methods of enhanced target engagement comprising providing a KRAS4A^{G34T} variant nucleotide sequence (e.g., a variant of SEQ ID NO: 3) that encodes and active KRAS4A^{G12C} variant.

In some embodiments, provided herein are systems for enhanced target engagement comprising an active variant of KRAS4A^{G12D} (SEQ ID NO: 6). In some embodiments, active variants of KRAS4A^{G12D} are provided, for example, active variants (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 6. In some embodiments, an active KRAS4A^{G12D} variant is provided with one or more substitutions (e.g., 1, 2, 3, 4, 5, 6, 7, 8, 9, 10, 15, 20, 25, 30, 40, 50, 60, or ranges therebetween) relative to SEQ ID NO: 6. In some embodiments, an active KRAS4A^{G12D} variant comprises a D at position 12. In some embodiments, provided herein are methods for enhanced target engagement comprising in which active variants of KRAS4A^{G12D} (SEQ ID NO: 6) are provided and/or expressed.

In some embodiments, provided herein are systems for enhanced target engagement comprising a nucleic acid (e.g., variants of SEQ ID NO: 5) encoding an active variant of KRAS4A^{G12D}. In some embodiments, sequences encoding active variants of KRAS4A^{G12D} are provided, for example, sequences encoding active variants (e.g., constitutively active) comprising at least 70% (e.g., 70%, 75%, 80%, 85%, 90%, 95%, 96%, 97%, 98%, 99%, 100%, or ranges therebetween) sequence identity with the KRAS4A^{G35A} sequence SEQ ID NO: 5. In some embodiments, sequences comprising at least 70% (e.g., 70%, 75%, 80%, 85%, 90%, 95%, 96%, 97%, 98%, 99%, 100%, or ranges therebetween) sequence identity with SEQ ID NO: 5 are provided. In some embodiments, a KRAS4A^{G35A} variant nucleotide sequence is provided with one or more substitutions (e.g., 1, 2, 3, 4, 5, 6, 7, 8, 9, 10, 15, 20, 25, 30, 40, 50, 60, or ranges therebetween) relative to SEQ ID NO: 5. In some embodiments, a KRAS4A^{G35A} variant nucleotide sequence comprises a A at position 35 of SEQ ID NO: 5. In some embodiments, provided herein are methods of enhanced target engagement comprising providing a KRAS4A^{G35A} variant nucleotide sequence (e.g., a variant of SEQ ID NO: 5) that encodes and active KRAS4A^{G12D} variant.

In some embodiments, provided herein are systems for enhanced target engagement comprising an active variant of KRAS4A^{G12V} (SEQ ID NO: 8). In some embodiments, active variants of KRAS4A^{G12V} are provided, for example, active variants (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 8. In some embodiments, an active KRAS4A^{G12V} variant is provided with one or more substitutions (e.g., 1, 2, 3, 4, 5, 6, 7, 8, 9, 10, 15, 20, 25, 30, 40, 50, 60, or ranges therebetween) relative to SEQ ID NO: 8. In some embodiments, an active KRAS4A^{G12V} variant comprises a V at position 12. In some embodiments, provided herein are methods for enhanced target engagement comprising in which active variants of KRAS4A^{G12V} (SEQ ID NO: 8) are provided and/or expressed.

In some embodiments, provided herein are systems for enhanced target engagement comprising a nucleic acid (e.g., variants of SEQ ID NO: 7) encoding an active variant of KRAS4A^{G12V}. In some embodiments, sequences encoding active variants of KRAS4A^{G12V} are provided, for example, sequences encoding active variants (e.g., constitutively active) comprising at least 70% (e.g., 70%, 75%, 80%, 85%, 90%, 95%, 96%, 97%, 98%, 99%, 100%, or ranges therebetween) sequence identity with the KRAS4A^{G35T} sequence SEQ ID NO: 7. In some embodiments, sequences comprising at least 70% (e.g., 70%, 75%, 80%, 85%, 90%, 95%, 96%, 97%, 98%, 99%, 100%, or ranges therebetween) sequence identity with SEQ ID NO: 7 are provided. In some embodiments, a KRAS4A^{G35T} variant nucleotide sequence is provided with one or more substitutions (e.g., 1, 2, 3, 4, 5, 6, 7, 8, 9, 10, 15, 20, 25, 30, 40, 50, 60, or ranges therebetween) relative to SEQ ID NO: 7. In some embodiments, a KRAS4A^{G35T} variant nucleotide sequence comprises a T at position 35 of SEQ ID NO: 7. In some embodiments, provided herein are methods of enhanced target engagement comprising providing a KRAS4A^{G35T} variant nucleotide sequence (e.g., a variant of SEQ ID NO: 5) that encodes and active KRAS4A^{G12V} variant.

In some embodiments, provided herein are systems for enhanced target engagement comprising an active variant of KRAS4B (SEQ ID NO: 10). In some embodiments, active variants of KRAS4B are provided, for example, active variants (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 10. In some embodiments, an active KRAS4B variant is provided with one or more substitutions (e.g., 1, 2, 3, 4, 5, 6, 7, 8, 9, 10, 15, 20, 25, 30, 40, 50, 60, or ranges therebetween) relative to SEQ ID NO: 10. In some embodiments, an active KRAS4B variant comprises a substitution at position 12. In some embodiments, provided herein are methods for enhanced target engagement comprising in which active variants of KRAS4B (SEQ ID NO: 10) are provided and/or expressed.

In some embodiments, provided herein are systems for enhanced target engagement comprising a nucleic acid (e.g., variants of SEQ ID NO: 9) encoding an active variant of KRAS4B. In some embodiments, sequences encoding active variants of KRAS4B are provided, for example, sequences encoding active variants (e.g., constitutively active) comprising at least 70% (e.g., 70%, 75%, 80%, 85%, 90%, 95%, 96%, 97%, 98%, 99%, 100%, or ranges therebetween) sequence identity with the KRAS4B sequence SEQ ID NO: 9. In some embodiments, sequences comprising at least 70% (e.g., 70%, 75%, 80%, 85%, 90%, 95%, 96%, 97%, 98%, 99%, 100%, or ranges therebetween) sequence identity with SEQ ID NO: 9 are provided. In some embodiments, a KRAS4B variant nucleotide sequence is provided with one or more substitutions (e.g., 1, 2, 3, 4, 5, 6, 7, 8, 9, 10, 15, 20, 25, 30, 40, 50, 60, or ranges therebetween) relative to SEQ ID NO: 9. In some embodiments, a KRAS4B variant nucleotide sequence comprises a substitution at one or more of positions 34, 35 or 36 of SEQ ID NO: 9. In some embodiments, provided herein are methods of enhanced target engagement comprising providing a KRAS4B variant nucleotide sequence (e.g., a variant of SEQ ID NO: 1) that encodes and active KRAS4B variant.

In some embodiments, provided herein are systems for enhanced target engagement comprising an active variant of KRAS4B^{G12C} (SEQ ID NO: 12). In some embodiments, active variants of KRAS4B^{G12C} are provided, for example, active variants (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 12. In some embodiments, an active KRAS4B^{G12C} variant is provided with one or more substitutions (e.g., 1, 2, 3, 4, 5, 6, 7, 8, 9, 10, 15, 20, 25, 30, 40, 50, 60, or ranges therebetween) relative to SEQ ID NO: 12. In some embodiments, an active KRAS4B^{G12C} variant comprises a C at position 12. In some embodiments, provided herein are methods for enhanced target engagement comprising in which active variants of KRAS4B^{G12C} (SEQ ID NO: 12) are provided and/or expressed.

In some embodiments, provided herein are systems for enhanced target engagement comprising a nucleic acid (e.g., variants of SEQ ID NO: 11) encoding an active variant of KRAS4B^{G12C}. In some embodiments, sequences encoding active variants of KRAS4B^{G12C} are provided, for example, sequences encoding active variants (e.g., constitutively active) comprising at least 70% (e.g., 70%, 75%, 80%, 85%, 90%, 95%, 96%, 97%, 98%, 99%, 100%, or ranges therebetween) sequence identity with the KRAS4B^{G34T} sequence SEQ ID NO: 11. In some embodiments, sequences comprising at least 70% (e.g., 70%, 75%, 80%, 85%, 90%, 95%, 96%, 97%, 98%, 99%, 100%, or ranges therebetween) sequence identity with SEQ ID NO: 11 are provided. In some embodiments, a KRAS4B^{G34T} variant nucleotide sequence is provided with one or more substitutions (e.g., 1, 2, 3, 4, 5, 6, 7, 8, 9, 10, 15, 20, 25, 30, 40, 50, 60, or ranges therebetween) relative to SEQ ID NO: 11. In some embodiments, a KRAS4B^{G34T} variant nucleotide sequence comprises a T at position 34 of SEQ ID NO: 11. In some embodiments, provided herein are methods of enhanced target engagement comprising providing a KRAS4B^{G34T} variant nucleotide sequence (e.g., a variant of SEQ ID NO: 11) that encodes and active KRAS4B^{G12C} variant.

In some embodiments, provided herein are systems for enhanced target engagement comprising an active variant of KRAS4B^{G12D} (SEQ ID NO: 14). In some embodiments, active variants of KRAS4B^{G12D} are provided, for example, active variants (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 14. In some embodiments, an active KRAS4B^{G12D} variant is provided with one or more substitutions (e.g., 1, 2, 3, 4, 5, 6, 7, 8, 9, 10, 15, 20, 25, 30, 40, 50, 60, or ranges therebetween) relative to SEQ ID NO: 14. In some embodiments, an active KRAS4B^{G12D} variant comprises a D at position 12. In some embodiments, provided herein are methods for enhanced target engagement comprising in which active variants of KRAS4B^{G12D} (SEQ ID NO: 14) are provided and/or expressed.

In some embodiments, provided herein are systems for enhanced target engagement comprising a nucleic acid (e.g., variants of SEQ ID NO: 13) encoding an active variant of KRAS4B^{G12D}. In some embodiments, sequences encoding active variants of KRAS4B^{G12D} are provided, for example, sequences encoding active variants (e.g., constitutively active) comprising at least 70% (e.g., 70%, 75%, 80%, 85%, 90%, 95%, 96%, 97%, 98%, 99%, 100%, or ranges therebetween) sequence identity with the KRAS4B^{G35A} sequence SEQ ID NO: 13. In some embodiments, sequences comprising at least 70% (e.g., 70%, 75%, 80%, 85%, 90%, 95%, 96%, 97%, 98%, 99%, 100%, or ranges therebetween) sequence identity with SEQ ID NO: 13 are provided. In some embodiments, a KRAS4B^{G35A} variant nucleotide sequence is provided with one or more substitutions (e.g., 1, 2, 3, 4, 5, 6, 7, 8, 9, 10, 15, 20, 25, 30, 40, 50, 60, or ranges therebetween) relative to SEQ ID NO: 13. In some embodiments, a KRAS4B^{G35A} variant nucleotide sequence comprises an A at position 35 of SEQ ID NO: 13. In some embodiments, provided herein are methods of enhanced target engagement comprising providing a KRAS4B^{G35A} variant nucleotide sequence (e.g., a variant of SEQ ID NO: 13) that encodes and active KRAS4B^{G12D} variant.

In some embodiments, provided herein are systems for enhanced target engagement comprising an active variant of KRAS4B^{G12V} (SEQ ID NO: 16). In some embodiments, active variants of KRAS4B^{G12V} are provided, for example, active variants (e.g., constitutively active) comprising at least 70% (e.g., 70%, 75%, 80%, 85%, 90%, 95%, 96%, 97%, 98%, 99%, 100%, or ranges therebetween) sequence identity with SEQ ID NO: 16. In some embodiments, an active KRAS4B^{G12V} variant is provided with one or more substitutions (e.g., 1, 2, 3, 4, 5, 6, 7, 8, 9, 10, 15, 20, 25, 30, 40, 50, 60, or ranges therebetween) relative to SEQ ID NO: 16. In some embodiments, an active KRAS4B^{G12V} variant comprises a V at position 12. In some embodiments, provided herein are methods for enhanced target engagement comprising in which active variants of KRAS4B^{G12V} (SEQ ID NO: 16) are provided and/or expressed.

### II. Functional Kinase Binding Agents

In some embodiments, the kinase binding agent is general kinase inhibitor or a specific kinase inhibitor (e.g., a drug molecule that binds to and inhibits one or more kinases). Exemplary kinase inhibitors that find use as kinase binding moieties in embodiments herein include, but are not limited to afatinib, nintedanib, crizotinib, alectinib, trametinib, cabozantinib, midostaurin, dabrafenib, sunitinib, ruxolitinib, vemurafenib, sorafenib, axitinib, lenvatinib, regorafenib, ponatinib, cabozantinib, brigatinib, avapritinib, erdafitinib, encorafenib, vandetanib, cobimetinib, fedratinib, selumetinib, lorlatinib, binimetinib, entrectinib, pexidartinib, larotrectinib, gilteritinib, and ceritinib.

In some embodiments, provided herein are functional kinase binding agents comprising a kinase binding moiety linked to a functional element, such as:

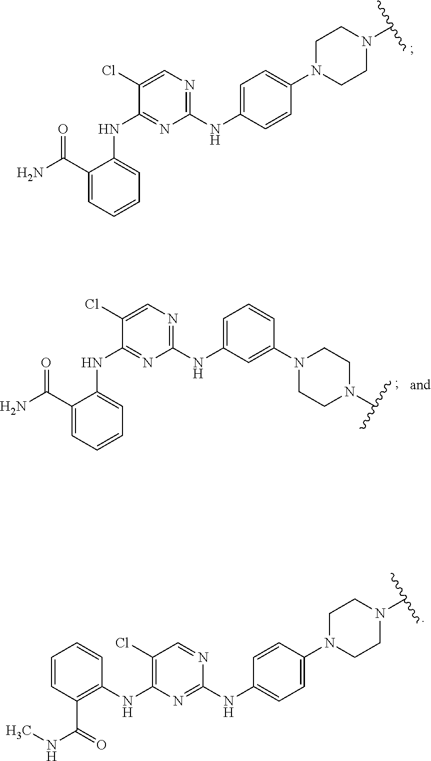 [SMILES: *N1CCN(c2ccc(Nc3ncc(Cl)c(Nc4ccccc4C(N)=O)n3)cc2)CC1 ; *N1CCN(c2cccc(Nc3ncc(Cl)c(Nc4ccccc4C(N)=O)n3)c2)CC1 ; *N1CCN(c2ccc(Nc3ncc(Cl)c(Nc4ccccc4C(=O)NC)n3)cc2)CC1]

In certain embodiments, a functional kinase binding agent comprises any ligand capable of binding (e.g., stably) to a kinase tethered to a functional element.

In some embodiments, a linker provides sufficient distance between the kinase binding moiety and the functional element (e.g., detectable element, capture element, affinity element, solid surface, etc.) to allow each to function undisturbed (or minimally disturbed by the linkage to the other. For example, linkers provide sufficient distance to allow a kinase binding agent to bind a kinase and detectable moiety to be detectable (e.g., without or with minimal interference between the two). In some embodiments, a linker separates a compound herein (e.g., CC-1852, CC-1861, CC-CTx-0294885, analogs or derivatives thereof (e.g., CC-1816, CC-1817, CC-1803, CC-1804, CC-1290, CC1294, etc.), etc.) and a detectable element (e.g., detectable element, solid surface, etc.) by 5 angstroms to 1000 angstroms, inclusive, in length. Suitable linkers separate a compound herein and a detectable element by 5 Å, 10 Å, 20 Å, 50 Å, 100 Å, 150 Å, 200 Å, 300 Å, 400 Å, 500 Å, 600 Å, 700 Å, 800 Å, 900 Å, 1000 Å, and any suitable ranges therein (e.g., 5-100 Å, 50-500 Å, 150-700 Å, etc.). In some embodiments, the linker separates a compound herein and a detectable element by 1-200 atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therein (e.g., 2-20, 10-50, etc.)).

In some embodiments, a linker comprises 1 or more (e.g., 1-20 (e.g., 1, 2, 3, 4, 5, 6, 7, 8, 9, 10, 11, 12, 13, 14, 15, 16, 17, 18, 19, 20, or any ranges therebetween) —(CH₂)₂O-(oxyethylene) groups (e.g., —(CH₂)₂O—(CH₂)₂O—(CH₂)₂O—(CH₂)₂O—, —(CH₂)₂O—(CH₂)₂O—(CH₂)₂O—(CH₂)₂O— CH₂)₂O—, —(CH₂)₂O—(CH₂)₂O—(CH₂)₂O—(CH₂)₂O— CH₂)₂O—(CH₂)₂O—, etc.). In some embodiments, the linker is —(CH₂)₂O—(CH₂)₂O—(CH₂)₂O—(CH₂)₂O—.

In some embodiments, a linker is attached to a kinase binding moiety herein at the 4-position of a piperazine. In some embodiments, the N at the 4-position of the piperazine of a kinase binding moiety forms an amide bond with the terminus of a linker. In some embodiments, a linker comprises one or more (e.g., 2, 3, 4, 5, 6, or more or ranges therebetween) amides.

In some embodiments, a linker comprises two or more “linker moieties” (L¹, L², etc.). In some embodiments, a linker comprises a cleavable (e.g., enzymatically cleavable, chemically cleavable, etc.) moiety (Y) and 0, 1, 2, of more “linker moieties” (L¹, L², etc.). In some embodiments, linker moieties are straight or branched chains comprising any combination of alkyl, alkenyl, or alkynyl chains, and main-chain heteroatoms (e.g., O, S, N, P, etc.). In some embodiments, linker moieties comprises one or more backbone groups selected from of: —O—, —S—, —CH═CH—, ═C═, a carbon-carbon triple bond, C═O, NH, SH, OH, CN, etc. In some embodiments, a linker moiety comprises one or more substituents, pendants, side chains, etc., comprising any suitable organic functional groups (e.g., OH, NH₂, CN, ═O, SH, halogen (e.g., Cl, Br, F, I), COOH, CH₃, etc.).

In particular embodiments, a linker moiety comprises an alkyl carbamate group (e.g., (CH₂)ₙOCONH, (CH₂)ₙNHCOO, etc.). In some embodiments, the alkyl carbamate is oriented such the COO end is oriented toward the kinase binding moiety and the NH end is oriented toward the functional element. In some embodiments, the alkyl carbamate is oriented such the NH end is oriented toward the kinase binding moiety and the COO end is oriented toward the functional element. In some embodiments, a linker or linker moiety comprises a single alkyl carbamate group. In some embodiments, a linker or linker moiety comprises two or more alkyl carbamate groups (e.g., 2, 3, 4, 5, 6, 7, 8, etc.).

In some embodiments, a linker moiety comprises more than 1 linearly connected C, S, N, and/or O atoms. In some embodiments, a linker moiety comprises one or more alkyl carbamate groups. In some embodiments, a linker moiety comprises one or more alkyl groups (e.g., methyl, ethyl, propyl, butyl, pentyl, hexyl, etc.). In some embodiments, a linker moiety comprises 1-200 linearly connected atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therein (e.g., 2-20, 10-50, 6-18)). In some embodiments, a linker moiety is 1-200 linearly connected atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therein (e.g., 2-20, 10-50, 6-18)) in length.

In some embodiments, functional kinase binding agents comprise a kinase binding moiety linked (e.g., directly or via a linker) to a functional element (e.g., detectable element, capture element, affinity element, solid surface, etc.).

In some embodiments, a functional kinase binding agent is biocompatible (e.g., cell compatible) and/or cell permeable. Therefore, in some embodiments, suitable functional elements (e.g., detectable elements, affinity elements, solid supports, capture elements) are ones that are cell compatible and/or cell permeable within the context of such compositions. In some embodiments, a composition comprising an addition element, when added extracellularly, is capable of crossing the cell membrane to enter a cell (e.g., via diffusion, endocytosis, active transport, passive transport, etc.). In some embodiments, suitable functional elements and linkers are selected based on cell compatibility and/or cell permeability, in addition to their particular function.

In certain embodiments, functional elements have a detectable property that allows for detection of the functional kinase binding agent and/or an analyte (e.g., kinase) bound thereto. Detectable elements include those with a characteristic electromagnetic spectral property such as emission or absorbance, magnetism, electron spin resonance, electrical capacitance, dielectric constant, or electrical conductivity as well as functional groups which are ferromagnetic, paramagnetic, diamagnetic, luminescent, electrochemiluminescent, fluorescent, phosphorescent, chromatic, antigenic, or have a distinctive mass. A detectable element includes, but is not limited to, a nucleic acid molecule (e.g., DNA or RNA (e.g., an oligonucleotide or nucleotide), a protein (e.g., a luminescent protein, a peptide, a contrast agent (e.g., MRI contract agent), a radionuclide, an affinity tag (e.g., biotin or streptavidin), a hapten, an amino acid, a lipid, a lipid bilayer, a solid support, a fluorophore, a chromophore, a reporter molecule, a radionuclide, an electron opaque molecule, a MRI contrast agent (e.g., manganese, gadolinium(III), or iron-oxide particles), or a coordinator thereof, and the like. Methods to detect a particular detectable element, or to isolate a composition comprising a particular detectable element and anything bound thereto, are understood.

In some embodiments, a functional element is or comprises a solid support. Suitable solid supports include a sedimental particle such as a magnetic particle, a sepharose, or cellulose bead; a membrane; glass, e.g., glass slides; cellulose, alginate, plastic, or other synthetically prepared polymer (e.g., an Eppendorf tube or a well of a multi-well plate); self-assembled monolayers; a surface plasmon resonance chip; or a solid support with an electron conducting surface; etc.

Exemplary functional elements include haptens (e.g., molecules useful to enhance immunogenicity such as keyhole limpet hemacyanin), cleavable labels (e.g., photocleavable biotin) and fluorescent labels (e.g., N-hydroxysuccinimide (NHS) modified coumarin and succinimide or sulfonosuccinimide modified BODIPY (which can be detected by UV and/or visible excited fluorescence detection), rhodamine (R110, rhodols, CRG6, Texas Methyl Red (TAMRA), Rox5, FAM, or fluorescein), coumarin derivatives (e.g., 7 aminocoumarin, and 7-hydroxycoumarin, 2-amino-4-methoxynapthalene, 1-hydroxypyrene, resorufin, phenalenones or benzphenalenones (U.S. Pat. No. 4,812,409)), acridinones (U.S. Pat. No. 4,810,636), anthracenes, and derivatives of alpha and beta-naphthol, fluorinated xanthene derivatives including fluorinated fluoresceins and rhodols (e.g., U.S. Pat. No. 6,162,931), and bioluminescent molecules (e.g., luciferase (e.g., Oplophorus-derive luciferase (See e.g., U.S. application Ser. No. 12/773,002; U.S. application Ser. No. 13/287,986; herein incorporated by reference in their entireties) or GFP or GFP derivatives). A fluorescent (or bioluminescent) detectable element may be used to sense changes in a system, like phosphorylation, in real-time. A fluorescent molecule, such as a chemosensor of metal ions, may be employed to label proteins which bind the composition. A bioluminescent or fluorescent functional group such as BODIPY, rhodamine green, GFP, or infrared dyes, finds use as a detectable element and may, for instance, be employed in interaction studies (e.g., using BRET, FRET, LRET or electrophoresis).

Another class of detectable elements includes molecules detectable using electromagnetic radiation and includes, but is not limited to, xanthene fluorophores, dansyl fluorophores, coumarins and coumarin derivatives, fluorescent acridinium moieties, benzopyrene-based fluorophores as well as 7-nitrobenz-2-oxa-1,3-diazole, and 3-N-(7-nitrobenz-2-oxa-1,3-diazol-4-yl)-2,3-diamino-propionic acid. Preferably, the fluorescent molecule has a high quantum yield of fluorescence at a wavelength different from native amino acids and more preferably has high quantum yield of fluorescence that can be excited in the visible, or in both the UV and visible, portion of the spectrum. Upon excitation at a preselected wavelength, the molecule is detectable at low concentrations either visually or using conventional fluorescence detection methods. Electrochemiluminescent molecules such as ruthenium chelates and its derivatives or nitroxide amino acids and their derivatives are detectable at femtomolar ranges and below.

In some embodiments, a detectable element is a fluorophore. Suitable fluorophores for linking to a kinase binding moiety (e.g., to form a fluorescent tracer) include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLuoR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc. In some embodiments, a fluorophore is a rhodamine analog (e.g., carboxy rhodamine analog) such as those described in U.S. patent application Ser. No. 13/682,589, herein incorporated by reference in its entirety.

In addition to fluorescent molecules, a variety of molecules with physical properties based on the interaction and response of the molecule to electromagnetic fields and radiation find use in the compositions and methods described herein. These properties include absorption in the UV, visible, and infrared regions of the electromagnetic spectrum, presence of chromophores that are Raman active and can be further enhanced by resonance Raman spectroscopy, electron spin resonance activity, and nuclear magnetic resonances and molecular mass, e.g., via a mass spectrometer.

In some embodiments, a functional element is a capture element. In some embodiments, a capture element is a substrate for a protein (e.g., enzyme), and the capture agent is that protein. In some embodiments, a capture element is a “covalent substrate” or one that forms a covalent bond with a protein or enzyme that it reacts with. The substrate may comprise a reactive group (e.g., a modified substrate) that forms a covalent bond with the enzyme upon interaction with the enzyme, or the enzyme may be a mutant version that is unable to reconcile a covalently bound intermediate with the substrate. In some embodiments, the substrate is recognized by a mutant protein (e.g., mutant dehalogenase), which forms a covalent bond thereto. In such embodiments, while the interaction of the substrate and a wild-type version of the protein (e.g., dehalogenase) results in a product and the regeneration of the wild-type protein, interaction of the substrate (e.g., haloalkane) with the mutant version of the protein (e.g., dehalogenase) results in stable bond formation (e.g., covalent bond formation) between the protein and substrate. The substrate may be any suitable substrate for any mutant protein that has been altered to form an ultra-stable or covalent bond with its substrate that would ordinarily only transiently bound by the protein. In some embodiments, the protein is a mutant hydrolase or dehalogenase. In some embodiments, the protein is a mutant dehalogenase and the substrate is a haloalkane. In some embodiments, the haloalkane comprises an alkane (e.g., C₂-C₂₀) capped by a terminal halogen (e.g., Cl, Br, F, I, etc.). In some embodiments, the haloalkane is of the formula A-X, wherein X is a halogen (e.g., Cl, Br, F, I, etc.), and wherein A is an alkane comprising 2-20 carbons. In certain embodiments, A comprises a straight-chain segment of 2-12 carbons. In certain embodiments, A is a straight-chain segment of 2-12 carbons. In some embodiments, the haloalkane may comprise any additional pendants or substitutions that do not interfere with interaction with the mutant dehalogenase.

In some embodiments, a capture agent is a SNAP-Tag and a capture element is benzyl guanine (See, e.g., Crivat G, Taraska J W (January 2012). Trends in Biotechnology 30 (1): 8-16; herein incorporated by reference in its entirety). In some embodiments, a capture agent is a CLIP-Tag and a capture element is benzyl cytosine (See, e.g., Gautier, et al. Chem Biol. 2008 February; 15(2):128-36; herein incorporated by reference in its entirety).

Systems comprising mutant proteins (e.g., mutant hydrolases (e.g., mutant dehalogenases) that covalently bind their substrates (e.g., haloalkane substrates) are described, for example, in U.S. Pat. Nos. 7,238,842; 7,425,436; 7,429,472; 7,867,726; each of which is herein incorporated by reference in their entireties.

In some embodiments, a functional element of a functional kinase binding agent is an affinity element (e.g., that binds to an affinity agent). Examples of such pairs would include: an antibody as the affinity agent and an antigen as the affinity element; a His-tag as the affinity element and a nickel column as the affinity agent; a protein and small molecule with high affinity as the affinity agent and affinity element, respectively (e.g., streptavidin and biotin), etc. Examples of affinity molecules include molecules such as immunogenic molecules (e.g., epitopes of proteins, peptides, carbohydrates, or lipids (e.g., any molecule which is useful to prepare antibodies specific for that molecule)); biotin, avidin, streptavidin, and derivatives thereof; metal binding molecules; and fragments and combinations of these molecules. Exemplary affinity molecules include 5× His (HHHHH)(SEQ ID NO: 19), 6× His (HHHHHH)(SEQ ID NO: 20), C-myc (EQKLISEEDL) (SEQ ID NO: 21), Flag (DYKDDDDK) (SEQ ID NO: 22), SteptTag (WSHPQFEK)(SEQ ID NO: 23), HA Tag (YPYDVPDYA) (SEQ ID NO: 24), thioredoxin, cellulose binding domain, chitin binding domain, S-peptide, T7 peptide, calmodulin binding peptide, C-end RNA tag, metal binding domains, metal binding reactive groups, amino acid reactive groups, inteins, biotin, streptavidin, and maltose binding protein. Another example of an affinity molecule is dansyllysine. Antibodies that interact with the dansyl ring are commercially available (Sigma Chemical; St. Louis, Mo.) or can be prepared using known protocols such as described in Antibodies: A Laboratory Manual (Harlow and Lane, 1988).

### III. Kinases

Embodiments herein find use in the engagement of various kinases with a functional kinase binding agent.

In some embodiments, kinases are expressed endogenously in a sample (e.g., cell, cell lysate, cell-free system, tissue, organism, etc.). In some embodiments, kinases are expressed from a suitable genetic and/or viral vector (e.g., a vector introduced into the sample (e.g., cell)). Examples of viral vectors include, without limitation, vectors based on DNA or RNA viruses, such as adenovirus, adeno-associated virus (AAV), retroviruses, lentiviruses, vaccinia virus, measles viruses, herpes viruses, baculoviruses, and papilloma virus vectors. See, Kay et al., Proc. Natl. Acad. Sci. USA, 94:12744-12746 (1997) for a review of viral and non-viral vectors; incorporated by reference in its entirety. Examples of non-viral vectors include, without limitation, vectors based on plasmid DNA or RNA, retroelement, transposon, and episomal vectors.

In some embodiments, kinases are expressed/provided as a fusion and/or with a tag for detection, identification, etc. In some embodiments, kinases are expressed/provided as a fusion with a bioluminescent reporter. In some embodiments, kinases are expressed/provided as a fusion with a luciferase. In some embodiments, kinases are expressed/provided as a fusion with an active variant of an Oplophorus luciferase. In some embodiments, provided herein kinases a provided/expressed as fusions with bioluminescent polypeptides and/or components of bioluminescent complexes based on (e.g., structurally, functionally, etc.) the luciferase of Oplophorus gracilirostris, the NanoLuc® luciferase (Promega Corporation; U.S. Pat. Nos. 8,557,970; 8,669,103; herein incorporated by reference in their entireties), NanoBiT (U.S. Pat. No. 9,797,889; herein incorporated by reference in its entirety), or NanoTrip (U.S. patent application Ser. No. 16/439,565; and U.S. Prov. Appln. Ser. No. 62/941,255; both of which are herein incorporated by reference in their entireties). In some embodiments, methods and systems herein incorporate commercially available NanoLuc®-based technologies (e.g., NanoLuc® luciferase, NanoBRET, NanoBiT, NanoTrip, NanoGlo, etc.), but in other embodiments, various combinations, variations, or derivations from the commercially available NanoLuc®-based technologies are employed.

In some embodiments, kinases are expressed/provided as a fusion with a bioluminescent polypeptide including but not limited to NanoLuc® and/or the bioluminescent polypeptides described in PCT Appln. No. PCT/US2010/033449, U.S. Pat. No. 8,557,970, PCT Appln. No. PCT/2011/059018, and U.S. Pat. No. 8,669,103 (each of which is herein incorporated by reference in their entirety and for all purposes). In some embodiments, such bioluminescent polypeptides are linked (e.g., fused, chemically linked, etc.) to a kinase for use in the methods and systems described herein.

In some embodiments, kinases are expressed/provided as a fusion with a component of a bioluminescent complex, including but not limited to NanoBiT®, NanoTrip, and/or the peptide and polypeptide components of bioluminescent complexes described in, for example, PCT Appln. No. PCT/US14/26354; U.S. Pat. No. 9,797,889; U.S. patent application Ser. No. 16/439,565 (PCT/US2019/036844); and U.S. Prov. Appln. Ser. No. 62/941,255 (each of which is herein incorporated by reference in their entirety and for all purposes). In some embodiments, such peptide and/or polypeptide components of bioluminescent complexes are linked (e.g., fused, chemically linked, etc.) to a kinase for use in the methods and systems described herein.

As disclosed in PCT Appln. No. PCT/US13/74765 and U.S. patent application Ser. No. 15/263,416 (herein incorporated by reference in their entireties and for all purposes), a protein (e.g., kinase) that is linked (e.g., fused) to a bioluminescent reporter (e.g., luciferase, component of the bioluminescent complex, etc.) can be detected by bioluminescence resonance energy transfer (BRET) between the bioluminescent reporter and an energy acceptor (e.g., a fluorophore) present in the system or method and co-localized with the protein (e.g., kinase).

In some embodiments, provided herein are systems comprising kinases fused to bioluminescent reporters (e.g., NanoLuc®-based reporters) and functional kinase binding agents comprising an energy acceptor (e.g., a fluorophore) as the detectable element, wherein the emission spectrum of the bioluminescent reporter and the excitation spectrum of the fluorophore overlap, such that engagement (e.g., binding) of the functional kinase binding agent with to the kinase can be detected by an increase (e.g., the presence of) BRET between the bioluminescent reporter and the energy acceptor (e.g., a fluorophore).

In some embodiments, any of the NanoLuc®-based, NanoBiT-based, and/or NanoTrip-based peptides, polypeptide, complexes, fusions, and conjugates may find use in BRET-based applications with the systems and methods described herein. For example, in certain embodiments, provided herein is a kinase (or kinases) are fused to a bioluminescent reported (e.g., NanoLuc®-based, NanoBiT-based, and/or NanoTrip-based polypeptide, peptide, or complex), and a functional kinase binding agent comprising an energy acceptor (e.g., a fluorophore (e.g., fluorescent protein, small molecule fluorophore, etc.)), wherein the emission spectrum of the NanoLuc®-based, NanoBiT-based, and/or NanoTrip-based polypeptide, peptide, or complex overlaps the excitation spectrum of the energy acceptor (e.g., a fluorophore). In some embodiments, upon engagement of the functional kinase binding agent with the kinase, and in the presence of a substrate (e.g., coelenterazine, furimazine, etc.) for the bioluminescent reporter, BRET is detected.

As used herein, the term “energy acceptor” refers to any small molecule (e.g., chromophore), macromolecule (e.g., autofluorescent protein, phycobiliproteins, nanoparticle, surface, etc.), or molecular complex that produces a readily detectable signal in response to energy absorption (e.g., resonance energy transfer). In certain embodiments, an energy acceptor is a fluorophore or other detectable chromophore. Suitable fluorophores include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLuoR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc. In some embodiments, a fluorophore is a rhodamine analog (e.g., carboxy rhodamine analog), such as those described in U.S. patent application Ser. No. 13/682,589, herein incorporated by reference in its entirety.

In some embodiments, the systems and methods herein find use with a broad spectrum of kinases, including protein kinases are of the following common families or subgroups: AGC (e.g., containing the PKA, PKG and PKC subfamilies), CAMK (e.g., calcium/calmodulin-dependent protein kinases), CK1 (e.g., casein kinase 1), CMGC (e.g., containing the CDK, MAPK, GSK3 and CLK subfamilies), NEK, RGC (e.g., receptor guanylate cyclases), STE, TKL (e.g., tyrosine protein kinase-like), and Tyr (e.g., tyrosine protein kinase). In some embodiments, the functional kinase binding agents herein bind to one or more kinases of atypical kinase families, such as, ADCK, alpha-type, FAST, PDK/BCKDK, PI3/PI4-kinase, RIO-type, etc. In some embodiments, the functional kinase binding agents herein bind to kinases of any suitable organism. In some embodiments, systems and methods herein find use with human and/or mouse kinases, such as those listed in Tables 1A-O, and/or homologs and analogs from other organisms.

TABLE 1A
AGC Ser/Thr protein kinase family

| | | |
|---|---|---|
| AKT1 | AKT1_HUMAN (P31749) | AKT1_MOUSE (P31750) |
| AKT2 | AKT2_HUMAN (P31751) | AKT2_MOUSE (Q60823) |
| AKT3 | AKT3_HUMAN (Q9Y243) | AKT3_MOUSE (Q9WUA6) |
| CDC42BPA | MRCKA_HUMAN (Q5VT25) | MRCKA_MOUSE (Q3UU96) |
| CDC42BPB | MRCKB_HUMAN (Q9Y5S2) | MRCKB_MOUSE (Q7TT50) |
| CDC42BPG | MRCKG_HUMAN (Q6DT37) | MRCKG_MOUSE (Q80UW5) |
| CIT | CTRO_HUMAN (O14578) | CTRO_MOUSE (P49025) |
| DMPK | DMPK_HUMAN (Q09013) | DMPK_MOUSE (P54265) |
| GRK1 | RK_HUMAN (Q15835) | RK_MOUSE (Q9WVL4) |
| GRK2 | ARBK1_HUMAN (P25098) | ARBK1_MOUSE (Q99MK8) |
| GRK3 | ARBK2_HUMAN (P35626) | ARBK2_MOUSE (Q3UYH7) |
| GRK4 | GRK4_HUMAN (P32298) | GRK4_MOUSE (O70291) |
| GRK5 | GRK5_HUMAN (P34947) | GRK5_MOUSE (Q8VEB1) |
| GRK6 | GRK6_HUMAN (P43250) | GRK6_MOUSE (O70293) |
| GRK7 | GRK7_HUMAN (Q8WTQ7) | |
| LATS1 | LATS1_HUMAN (O95835) | LATS1_MOUSE (Q8BYR2) |
| LATS2 | LATS2_HUMAN (Q9NRM7) | LATS2_MOUSE (Q7TSJ6) |
| MAST1 | MAST1_HUMAN (Q9Y2H9) | MAST1_MOUSE (Q9R1L5) |
| MAST2 | MAST2_HUMAN (Q6P0Q8) | MAST2_MOUSE (Q60592) |
| MAST3 | MAST3_HUMAN (O60307) | MAST3_MOUSE (Q3U214) |
| MAST4 | MAST4_HUMAN (O15021) | MAST4_MOUSE (Q811L6) |
| MASTL | GWL_HUMAN (Q96GX5) | GWL_MOUSE (Q8C0P0) |
| PDPK1 | PDPK1_HUMAN (O15530) | PDPK1_MOUSE (Q9Z2A0) |
| PDPK2P | PDPK2_HUMAN (Q6A1A2) | |
| PKN1 | PKN1_HUMAN (Q16512) | PKN1_MOUSE (P70268) |
| PKN2 | PKN2_HUMAN (Q16513) | PKN2_MOUSE (Q8BWW9) |
| PKN3 | PKN3_HUMAN (Q6P5Z2) | PKN3_MOUSE (Q8K045) |
| PRKACA | KAPCA_HUMAN (P17612) | KAPCA_MOUSE (P05132) |
| PRKACB | KAPCB_HUMAN (P22694) | KAPCB_MOUSE (P68181) |
| PRKACG | KAPCG_HUMAN (P22612) | |
| PRKCA | KPCA_HUMAN (P17252) | KPCA_MOUSE (P20444) |
| PRKCB | KPCB_HUMAN (P05771) | KPCB_MOUSE (P68404) |
| PRKCD | KPCD_HUMAN (Q05655) | KPCD_MOUSE (P28867) |
| PRKCE | KPCE_HUMAN (Q02156) | KPCE_MOUSE (P16054) |
| PRKCG | KPCG_HUMAN (P05129) | KPCG_MOUSE (P63318) |
| PRKCH | KPCL_HUMAN (P24723) | KPCL_MOUSE (P23298) |
| PRKCI | KPCI_HUMAN (P41743) | KPCI_MOUSE (Q62074) |
| PRKCQ | KPCT_HUMAN (Q04759) | KPCT_MOUSE (Q02111) |
| PRKCZ | KPCZ_HUMAN (Q05513) | KPCZ_MOUSE (Q02956) |
| PRKG1 | KGP1_HUMAN (Q13976) | KGP1_MOUSE (P0C605) |
| PRKG2 | KGP2_HUMAN (Q13237) | KGP2_MOUSE (Q61410) |
| PRKX | PRKX_HUMAN (P51817) | PRKX_MOUSE (Q922R0) |
| PRKY | PRKY_HUMAN (O43930) | |
| ROCK1 | ROCK1_HUMAN (Q13464) | ROCK1_MOUSE (P70335) |
| ROCK2 | ROCK2_HUMAN (O75116) | ROCK2_MOUSE (P70336) |
| RPS6KA1 | KS6A1_HUMAN (Q15418) | KS6A1_MOUSE (P18653) |
| RPS6KA2 | KS6A2_HUMAN (Q15349) | KS6A2_MOUSE (Q9WUT3) |
| RPS6KA3 | KS6A3_HUMAN (P51812) | KS6A3_MOUSE (P18654) |
| RPS6KA4 | KS6A4_HUMAN (O75676) | KS6A4_MOUSE (Q9Z2B9) |
| RPS6KA5 | KS6A5_HUMAN (O75582) | KS6A5_MOUSE (Q8C050) |
| RPS6KA6 | KS6A6_HUMAN (Q9UK32) | KS6A6_MOUSE (Q7TPSO) |
| RPS6KB1 | KS6B1_HUMAN (P23443) | KS6B1_MOUSE (Q8BSK8) |
| RPS6KB2 | KS6B2_HUMAN (Q9UBS0) | KS6B2_MOUSE (Q9Z1M4) |
| SGK1 | SGK1_HUMAN (O00141) | SGK1_MOUSE (Q9WVC6) |
| SGK2 | SGK2_HUMAN (Q9HBY8) | SGK2_MOUSE (Q9QZS5) |
| SGK3 | SGK3_HUMAN (Q96BR1) | SGK3_MOUSE (Q9ERE3) |
| STK38 | STK38_HUMAN (Q15208) | STK38_MOUSE (Q91VJ4) |
| STK38L | ST38L_HUMAN (Q9Y2H1) | ST38L_MOUSE (Q7TSE6) |

TABLE 1B
CAMK Ser/Thr protein kinase family

| | | |
|---|---|---|
| | | SMKX_MOUSE (Q8C0X8) |
| BRSK1 | BRSK1_HUMAN (Q8TDC3) | BRSK1_MOUSE (Q5RJI5) |
| BRSK2 | BRSK2_HUMAN (Q8IWQ3) | BRSK2_MOUSE (Q69Z98) |
| CAMK1 | KCC1A_HUMAN (Q14012) | KCC1A_MOUSE (Q91YS8) |
| CAMK1D | KCC1D_HUMAN (Q8IU85) | KCC1D_MOUSE (Q8BW96) |
| CAMK1G | KCC1G_HUMAN (Q96NX5) | KCC1G_MOUSE (Q91VB2) |
| CAMK2A | KCC2A_HUMAN (Q9UQM7) | KCC2A_MOUSE (P11798) |
| CAMK2B | KCC2B_HUMAN (Q13554) | KCC2B_MOUSE (P28652) |
| CAMK2D | KCC2D_HUMAN (Q13557) | KCC2D_MOUSE (Q6PHZ2) |
| CAMK2G | KCC2G_HUMAN (Q13555) | KCC2G_MOUSE (Q923T9) |
| CAMK4 | KCC4_HUMAN (Q16566) | KCC4_MOUSE (P08414) |
| CAMKV | CAMKV_HUMAN (Q8NCB2) | CAMKV_MOUSE (Q3UHL1) |
| CASK | CSKP_HUMAN (O14936) | CSKP_MOUSE (O70589) |
| CHEK1 | CHK1_HUMAN (O14757) | CHK1_MOUSE (O35280) |
| CHEK2 | CHK2_HUMAN (O96017) | CHK2_MOUSE (Q9Z265) |
| DAPK1 | DAPK1_HUMAN (P53355) | DAPK1_MOUSE (Q80YE7) |
| DAPK2 | DAPK2_HUMAN (Q9UIK4) | DAPK2_MOUSE (Q8VDF3) |
| DAPK3 | DAPK3_HUMAN (O43293) | DAPK3_MOUSE (O54784) |
| DCLK1 | DCLK1_HUMAN (O15075) | DCLK1_MOUSE (Q9JLM8) |
| DCLK2 | DCLK2_HUMAN (Q8N568) | DCLK2_MOUSE (Q6PGN3) |
| DCLK3 | DCLK3_HUMAN (Q9C098) | DCLK3_MOUSE (Q8BWQ5) |
| Gm4922 | | SMKZ_MOUSE (Q8C0N0) |
| Gm7168 | | SMKY_MOUSE (A0AUV4) |
| HUNK | HUNK_HUMAN (P57058) | HUNK_MOUSE (O88866) |
| KALRN | KALRN_HUMAN (O60229) | KALRN_MOUSE (A2CG49) |
| MAPKAPK2 | MAPK2_HUMAN (P49137) | MAPK2_MOUSE (P49138) |
| MAPKAPK3 | MAPK3_HUMAN (Q16644) | MAPK3_MOUSE (Q3UMW7) |
| MAPKAPK5 | MAPK5_HUMAN (Q8IW41) | MAPK5_MOUSE (O54992) |
| MARK1 | MARK1_HUMAN (Q9P0L2) | MARK1_MOUSE (Q8VHJ5) |
| MARK2 | MARK2_HUMAN (Q7KZI7) | MARK2_MOUSE (Q05512) |
| MARK3 | MARK3_HUMAN (P27448) | MARK3_MOUSE (Q03141) |
| MARK4 | MARK4_HUMAN (Q96L34) | MARK4_MOUSE (Q8CIP4) |
| MELK | MELK_HUMAN (Q14680) | MELK_MOUSE (Q61846) |
| MKNK1 | MKNK1_HUMAN (Q9BUB5) | MKNK1_MOUSE (O08605) |
| MKNK2 | MKNK2_HUMAN (Q9HBH9) | MKNK2_MOUSE (Q8CDB0) |
| MYLK | MYLK_HUMAN (Q15746) | MYLK_MOUSE (Q6PDN3) |
| MYLK2 | MYLK2_HUMAN (Q9H1R3) | MYLK2_MOUSE (Q8VCR8) |
| MYLK3 | MYLK3_HUMAN (Q32MK0) | MYLK3_MOUSE (Q3UIZ8) |
| MYLK4 | MYLK4_HUMAN (Q86YV6) | MYLK4_MOUSE (Q5SUV5) |
| NIM1K | NIM1_HUMAN (Q8IY84) | NIM1_MOUSE (Q8BHI9) |
| NUAK1 | NUAK1_HUMAN (O60285) | NUAK1_MOUSE (Q641K5) |
| NUAK2 | NUAK2_HUMAN (Q9H093) | NUAK2_MOUSE (Q8BZN4) |
| OBSCN | OBSCN_HUMAN (Q5VST9) | OBSCN_MOUSE (A2AAJ9) |
| PASK | PASK_HUMAN (Q96RG2) | PASK_MOUSE (Q8CEE6) |
| PHKG1 | PHKG1_HUMAN (Q16816) | PHKG1_MOUSE (P07934) |
| PHKG2 | PHKG2_HUMAN (P15735) | PHKG2_MOUSE (Q9DB30) |
| PIM1 | PIM1_HUMAN (P11309) | PIM1_MOUSE (P06803) |
| PIM2 | PIM2_HUMAN (Q9P1W9) | PIM2_MOUSE (Q62070) |
| PIM3 | PIM3_HUMAN (Q86V86) | PIM3_MOUSE (P58750) |
| PNCK | KCC1B_HUMAN (Q6P2M8) | KCC1B_MOUSE (Q9QYK9) |
| PRKAA1 | AAPK1_HUMAN (Q13131) | AAPK1_MOUSE (Q5EG47) |
| PRKAA2 | AAPK2_HUMAN (P54646) | AAPK2_MOUSE (Q8BRK8) |
| PRKD1 | KPCD1_HUMAN (Q15139) | KPCD1_MOUSE (Q62101) |
| PRKD2 | KPCD2_HUMAN (Q9BZL6) | KPCD2_MOUSE (Q8BZ03) |
| PRKD3 | KPCD3_HUMAN (O94806) | KPCD3_MOUSE (Q8K1Y2) |
| PSKH1 | KPSH1_HUMAN (P11801) | KPSH1_MOUSE (Q91YA2) |
| PSKH2 | KPSH2_HUMAN (Q96Q56) | |
| SIK1 | SIK1_HUMAN (P57059) | SIK1_MOUSE (Q60670) |
| SIK2 | SIK2_HUMAN (Q9H0K1) | SIK2_MOUSE (Q8CFH6) |
| SIK3 | SIK3_HUMAN (Q9Y2K2) | SIK3_MOUSE (Q6P456) |
| SNRK | SNRK_HUMAN (Q9NRH2) | SNRK_MOUSE (Q8VDU5) |
| SPEG | SPEG_HUMAN (Q15772) | SPEG_MOUSE (Q62407) |
| STK11 | STK11_HUMAN (Q15831) | STK11_MOUSE (Q9WTK7) |
| STK17A | ST17A_HUMAN (Q9UEE5) | |
| STK17B | ST17B_HUMAN (O94768) | ST17B_MOUSE (Q8BG48) |
| STK33 | STK33_HUMAN (Q9BYT3) | STK33_MOUSE (Q924X7) |
| STK40 | STK40_HUMAN (Q8N2I9) | STK40_MOUSE (Q7TNL3) |
| Smok2a | | SMK2A_MOUSE (Q9QYZ6) |
| Smok2b | | SMK2B_MOUSE (Q9QYZ3) |
| Smok3a | | SMK3A_MOUSE (C0HKC8) |
| Smok3b | | SMK3B_MOUSE (C0HKC9) |
| Stk-ps2 | | SMKW_MOUSE (Q8C0V7) |
| TRIB1 | TRIB1_HUMAN (Q96RU8) | TRIB1_MOUSE (Q8K4K4) |
| TRIB2 | TRIB2_HUMAN (Q92519) | TRIB2_MOUSE (Q8K4K3) |
| TRIB3 | TRIB3_HUMAN (Q96RU7) | TRIB3_MOUSE (Q8K4K2) |
| TRIO | TRIO_HUMAN (O75962) | TRIO_MOUSE (Q0KL02) |
| TSSK1B | TSSK1_HUMAN (Q9BXA7) | TSSK1_MOUSE (Q61241) |
| TSSK2 | TSSK2_HUMAN (Q96PF2) | TSSK2_MOUSE (O54863) |
| TSSK3 | TSSK3_HUMAN (Q96PN8) | TSSK3_MOUSE (Q9D2E1) |
| TSSK4 | TSSK4_HUMAN (Q6SA08) | TSSK4_MOUSE (Q9D411) |
| TSSK6 | TSSK6_HUMAN (Q9BXA6) | TSSK6_MOUSE (Q925K9) |
| TTN | TITIN_HUMAN (Q8WZ42) | TITIN_MOUSE (A2ASS6) |
| Tssk5 | | TSSK5_MOUSE (Q8C1R0) |

TABLE 1C
CK1 Ser/Thr protein kinase family

| | | |
|---|---|---|
| CSNK1A1 | KC1A_HUMAN (P48729) | KC1A_MOUSE (Q8BK63) |
| CSNK1A1L | KC1AL_HUMAN (Q8N752) | |
| CSNK1D | KC1D_HUMAN (P48730) | KC1D_MOUSE (Q9DC28) |
| CSNK1E | KC1E_HUMAN (P49674) | KC1E_MOUSE (Q9JMK2) |
| CSNK1G1 | KC1G1_HUMAN (Q9HCP0) | KC1G1_MOUSE (Q8BTH8) |
| CSNK1G2 | KC1G2_HUMAN (P78368) | KC1G2_MOUSE (Q8BVP5) |
| CSNK1G3 | KC1G3_HUMAN (Q9Y6M4) | KC1G3_MOUSE (Q8C4X2) |
| TTBK1 | TTBK1_HUMAN (Q5TCY1) | TTBK1_MOUSE (Q6PCN3) |
| TTBK2 | TTBK2_HUMAN (Q6IQ55) | TTBK2_MOUSE (Q3UVR3) |
| VRK1 | VRK1_HUMAN (Q99986) | VRK1_MOUSE (Q80X41) |
| VRK2 | VRK2_HUMAN (Q86Y07) | VRK2_MOUSE (Q8BN21) |
| VRK3 | VRK3_HUMAN (Q8IV63) | VRK3_MOUSE (Q8K3G5) |

TABLE 1D
CMGC Ser/Thr protein kinase family

| | | |
|---|---|---|
| CDK1 | CDK1_HUMAN (P06493) | CDK1_MOUSE (P11440) |
| CDK10 | CDK10_HUMAN (Q15131) | CDK10_MOUSE (Q3UMM4) |
| CDK11A | CD11A_HUMAN (Q9UQ88) | |
| CDK11B | CD11B_HUMAN (P21127) | CD11B_MOUSE (P24788) |
| CDK12 | CDK12_HUMAN (Q9NYV4) | CDK12_MOUSE (Q14AX6) |
| CDK13 | CDK13_HUMAN (Q14004) | CDK13_MOUSE (Q69ZA1) |
| CDK14 | CDK14_HUMAN (O94921) | CDK14_MOUSE (O35495) |
| CDK15 | CDK15_HUMAN (Q96Q40) | CDK15_MOUSE (Q3V3A1) |
| CDK16 | CDK16_HUMAN (Q00536) | CDK16_MOUSE (Q04735) |
| CDK17 | CDK17_HUMAN (Q00537) | CDK17_MOUSE (Q8K0D0) |
| CDK18 | CDK18_HUMAN (Q07002) | CDK18_MOUSE (Q04899) |
| CDK19 | CDK19_HUMAN (Q9BWU1) | CDK19_MOUSE (Q8BWD8) |
| CDK2 | CDK2_HUMAN (P24941) | CDK2_MOUSE (P97377) |
| CDK20 | CDK20_HUMAN (Q8IZL9) | CDK20_MOUSE (Q9JHU3) |
| CDK3 | CDK3_HUMAN (Q00526) | CDK3_MOUSE (Q80YP0) |
| CDK4 | CDK4_HUMAN (P11802) | CDK4_MOUSE (P30285) |
| CDK5 | CDK5_HUMAN (Q00535) | CDK5_MOUSE (P49615) |
| CDK6 | CDK6_HUMAN (Q00534) | CDK6_MOUSE (Q64261) |
| CDK7 | CDK7_HUMAN (P50613) | CDK7_MOUSE (Q03147) |
| CDK8 | CDK8_HUMAN (P49336) | CDK8_MOUSE (Q8R3L8) |
| CDK9 | CDK9_HUMAN (P50750) | CDK9_MOUSE (Q99J95) |
| CDKL1 | CDKL1_HUMAN (Q00532) | CDKL1_MOUSE (Q8CEQ0) |
| CDKL2 | CDKL2_HUMAN (Q92772) | CDKL2_MOUSE (Q9QUK0) |
| CDKL3 | CDKL3_HUMAN (Q8IVW4) | CDKL3_MOUSE (Q8BLF2) |
| CDKL4 | CDKL4_HUMAN (Q5MAI5) | CDKL4_MOUSE (Q3TZA2) |
| CDKL5 | CDKL5_HUMAN (O76039) | CDKL5_MOUSE (Q3UTQ8) |
| CLK1 | CLK1_HUMAN (P49759) | CLK1_MOUSE (P22518) |
| CLK2 | CLK2_HUMAN (P49760) | CLK2_MOUSE (O35491) |
| CLK3 | CLK3_HUMAN (P49761) | CLK3_MOUSE (O35492) |
| CLK4 | CLK4_HUMAN (Q9HAZ1) | CLK4_MOUSE (O35493) |
| DYRK1A | DYR1A_HUMAN (Q13627) | DYR1A_MOUSE (Q61214) |
| DYRK1B | DYR1B_HUMAN (Q9Y463) | DYR1B_MOUSE (Q9Z188) |
| DYRK2 | DYRK2_HUMAN (Q92630) | DYRK2_MOUSE (Q5U4C9) |
| DYRK3 | DYRK3_HUMAN (O43781) | DYRK3_MOUSE (Q922Y0) |
| DYRK4 | DYRK4_HUMAN (Q9NR20) | DYRK4_MOUSE (Q8BI55) |
| GSK3A | GSK3A_HUMAN (P49840) | GSK3A_MOUSE (Q2NL51) |
| GSK3B | GSK3B_HUMAN (P49841) | GSK3B_MOUSE (Q9WV60) |
| HIPK1 | HIPK1_HUMAN (Q86Z02) | HIPK1_MOUSE (O88904) |
| HIPK2 | HIPK2_HUMAN (Q9H2X6) | HIPK2_MOUSE (Q9QZR5) |
| HIPK3 | HIPK3_HUMAN (Q9H422) | HIPK3_MOUSE (Q9ERH7) |
| HIPK4 | HIPK4_HUMAN (Q8NE63) | HIPK4_MOUSE (Q3V016) |
| ICK | ICK_HUMAN (Q9UPZ9) | ICK_MOUSE (Q9JKV2) |
| MAK | MAK_HUMAN (P20794) | MAK_MOUSE (Q04859) |
| MAPK1 | MK01_HUMAN (P28482) | MK01_MOUSE (P63085) |
| MAPK10 | MK10_HUMAN (P53779) | MK10_MOUSE (Q61831) |
| MAPK11 | MK11_HUMAN (Q15759) | MK11_MOUSE (Q9WUI1) |
| MAPK12 | MK12_HUMAN (P53778) | MK12_MOUSE (O08911) |
| MAPK13 | MK13_HUMAN (O15264) | MK13_MOUSE (Q9Z1B7) |
| MAPK14 | MK14_HUMAN (Q16539) | MK14_MOUSE (P47811) |
| MAPK15 | MK15_HUMAN (Q8TD08) | MK15_MOUSE (Q80Y86) |
| MAPK3 | MK03_HUMAN (P27361) | MK03_MOUSE (Q63844) |
| MAPK4 | MK04_HUMAN (P31152) | MK04_MOUSE (Q6P5G0) |
| MAPK6 | MK06_HUMAN (Q16659) | MK06_MOUSE (Q61532) |
| MAPK7 | MK07_HUMAN (Q13164) | MK07_MOUSE (Q9WVS8) |
| MAPK8 | MK08_HUMAN (P45983) | MK08_MOUSE (Q91Y86) |
| MAPK9 | MK09_HUMAN (P45984) | MK09_MOUSE (Q9WTU6) |
| MOK | MOK_HUMAN (Q9UQ07) | MOK_MOUSE (Q9WVS4) |
| NLK | NLK_HUMAN (Q9UBE8) | NLK_MOUSE (O54949) |
| PRPF4B | PRP4B_HUMAN (Q13523) | PRP4B_MOUSE (Q61136) |
| SRPK1 | SRPK1_HUMAN (Q965B4) | SRPK1_MOUSE (O70551) |
| SRPK2 | SRPK2_HUMAN (P78362) | SRPK2_MOUSE (O54781) |
| SRPK3 | SRPK3_HUMAN (Q9UPE1) | SRPK3_MOUSE (Q9Z0G2) |

TABLE 1E
NEK Ser/Thr protein kinase family

| | | |
|---|---|---|
| NEK1 | NEK1_HUMAN (Q96PY6) | NEK1_MOUSE (P51954) |
| NEK10 | NEK10_HUMAN (Q6ZWH5) | NEK10_MOUSE (Q3UGM2) |
| NEK11 | NEK11_HUMAN (Q8NG66) | NEK11_MOUSE (Q8C0Q4) |
| NEK2 | NEK2_HUMAN (P51955) | NEK2_MOUSE (O35942) |
| NEK3 | NEK3_HUMAN (P51956) | NEK3_MOUSE (Q9R0A5) |
| NEK4 | NEK4_HUMAN (P51957) | NEK4_MOUSE (Q9Z1J2) |
| NEK5 | NEK5_HUMAN (Q6P3R8) | NEK5_MOUSE (Q7TSC3) |
| NEK6 | NEK6_HUMAN (Q9HC98) | NEK6_MOUSE (Q9ES70) |
| NEK7 | NEK7_HUMAN (Q8TDX7) | NEK7_MOUSE (Q9ES74) |
| NEK8 | NEK8_HUMAN (Q86SG6) | NEK8_MOUSE (Q91ZR4) |
| NEK9 | NEK9_HUMAN (Q8TD19) | NEK9_MOUSE (Q8K1R7) |

TABLE 1F
STE Ser/Thr protein kinase family

| | | |
|---|---|---|
| MAP2K1 | MP2K1_HUMAN (Q02750) | MP2K1_MOUSE (P31938) |
| MAP2K2 | MP2K2_HUMAN (P36507) | MP2K2_MOUSE (Q63932) |
| MAP2K3 | MP2K3_HUMAN (P46734) | MP2K3_MOUSE (O09110) |
| MAP2K4 | MP2K4_HUMAN (P45985) | MP2K4_MOUSE (P47809) |
| MAP2K5 | MP2K5_HUMAN (Q13163) | MP2K5_MOUSE (Q9WVS7) |
| MAP2K6 | MP2K6_HUMAN (P52564) | MP2K6_MOUSE (P70236) |
| MAP2K7 | MP2K7_HUMAN (O14733) | MP2K7_MOUSE (Q8CE90) |
| MAP3K1 | M3K1_HUMAN (Q13233) | M3K1_MOUSE (P53349) |
| MAP3K10 | M3K10_HUMAN (Q02779) | M3K10_MOUSE (Q66L42) |
| MAP3K11 | M3K11_HUMAN (Q16584) | M3K11_MOUSE (Q80XI6) |
| MAP3K12 | M3K12_HUMAN (Q12852) | M3K12_MOUSE (Q60700) |
| MAP3K13 | M3K13_HUMAN (O43283) | M3K13_MOUSE (Q1HKZ5) |
| MAP3K14 | M3K14_HUMAN (Q99558) | M3K14_MOUSE (Q9WUL6) |
| MAP3K15 | M3K15_HUMAN (Q6ZN16) | M3K15_MOUSE (A2AQW0) |
| MAP3K19 | M3K19_HUMAN (Q56UN5) | M3K19_MOUSE (E9Q354) |
| MAP3K2 | M3K2_HUMAN (Q9Y2U5) | M3K2_MOUSE (Q61083) |
| MAP3K20 | M3K20_HUMAN (Q9NYL2) | M3K20_MOUSE (Q9ESL4) |
| MAP3K21 | M3K21_HUMAN (Q5TCX8) | M3K21_MOUSE (Q8VDG6) |
| MAP3K3 | M3K3_HUMAN (Q99759) | M3K3_MOUSE (Q61084) |
| MAP3K4 | M3K4_HUMAN (Q9Y6R4) | M3K4_MOUSE (O08648) |
| MAP3K5 | M3K5_HUMAN (Q99683) | M3K5_MOUSE (O35099) |
| MAP3K6 | M3K6_HUMAN (O95382) | M3K6_MOUSE (Q9WTR2) |
| MAP3K7 | M3K7_HUMAN (O43318) | M3K7_MOUSE (Q62073) |
| MAP3K8 | M3K8_HUMAN (P41279) | M3K8_MOUSE (Q07174) |
| MAP3K9 | M3K9_HUMAN (P80192) | M3K9_MOUSE (Q3U1V8) |
| MAP4K1 | M4K1_HUMAN (Q92918) | M4K1_MOUSE (P70218) |
| MAP4K2 | M4K2_HUMAN (Q12851) | M4K2_MOUSE (Q61161) |
| MAP4K3 | M4K3_HUMAN (Q8IVH8) | M4K3_MOUSE (Q99JP0) |
| MAP4K4 | M4K4_HUMAN (O95819) | M4K4_MOUSE (P97820) |
| MAP4K5 | M4K5_HUMAN (Q9Y4K4) | M4K5_MOUSE (Q8BPM2) |
| MINK1 | MINK1_HUMAN (Q8N4C8) | MINK1_MOUSE (Q9JM52) |
| MYO3A | MYO3A_HUMAN (Q8NEV4) | MYO3A_MOUSE (Q8K3H5) |
| MYO3B | MYO3B_HUMAN (Q8WXR4) | MYO3B_MOUSE (Q1EG27) |
| NRK | NRK_HUMAN (Q7Z2Y5) | NRK_MOUSE (Q9R0G8) |
| OXSR1 | OXSR1_HUMAN (O95747) | OXSR1_MOUSE (Q6P9R2) |
| PAK1 | PAK1_HUMAN (Q13153) | PAK1_MOUSE (O88643) |
| PAK2 | PAK2_HUMAN (Q13177) | PAK2_MOUSE (Q8CIN4) |
| PAK3 | PAK3_HUMAN (O75914) | PAK3_MOUSE (Q61036) |
| PAK4 | PAK4_HUMAN (O96013) | PAK4_MOUSE (Q8BTW9) |
| PAK5 | PAK5_HUMAN (Q9P286) | PAK5_MOUSE (Q8C015) |
| PAK6 | PAK6_HUMAN (Q9NQU5) | PAK6_MOUSE (Q3ULB5) |
| PBK | TOPK_HUMAN (Q96KB5) | TOPK_MOUSE (Q9JJ78) |
| SLK | SLK_HUMAN (Q9H2G2) | SLK_MOUSE (O54988) |
| STK10 | STK10_HUMAN (O94804) | STK10_MOUSE (O55098) |
| STK24 | STK24_HUMAN (Q9Y6E0) | STK24_MOUSE (Q99KH8) |
| STK25 | STK25_HUMAN (O00506) | STK25_MOUSE (Q9Z2W1) |
| STK26 | STK26_HUMAN (Q9P289) | STK26_MOUSE (Q99JT2) |
| STK3 | STK3_HUMAN (Q13188) | STK3_MOUSE (Q9JI10) |
| STK39 | STK39_HUMAN (Q9UEW8) | STK39_MOUSE (Q9Z1W9) |
| STK4 | STK4_HUMAN (Q13043) | STK4_MOUSE (Q9JI11) |
| STRADA | STRAA_HUMAN (Q7RTN6) | STRAA_MOUSE (Q3UUJ4) |
| STRADB | STRAB_HUMAN (Q9C0K7) | STRAB_MOUSE (Q8K4T3) |
| TAOK1 | TAOK1_HUMAN (Q7L7X3) | TAOK1_MOUSE (Q5F2E8) |
| TAOK2 | TAOK2_HUMAN (Q9UL54) | TAOK2_MOUSE (Q6ZQ29) |
| TAOK3 | TAOK3_HUMAN (Q9H2K8) | TAOK3_MOUSE (Q8BYC6) |
| TNIK | TNIK_HUMAN (Q9UKE5) | TNIK_MOUSE (P83510) |

TABLE 1G
TKL Ser/Thr protein kinase family

| | | |
|---|---|---|
| ACVR1 | ACVR1_HUMAN (Q04771) | ACVR1_MOUSE (P37172) |
| ACVR1B | ACV1B_HUMAN (P36896) | ACV1B_MOUSE (Q61271) |
| ACVR1C | ACV1C_HUMAN (Q8NER5) | ACV1C_MOUSE (Q8K348) |
| ACVR2A | AVR2A_HUMAN (P27037) | AVR2A_MOUSE (P27038) |
| ACVR2B | AVR2B_HUMAN (Q13705) | AVR2B_MOUSE (P27040) |
| ACVRL1 | ACVL1_HUMAN (P37023) | ACVL1_MOUSE (Q61288) |
| AMHR2 | AMHR2_HUMAN (Q16671) | AMHR2_MOUSE (Q8K592) |
| ANKK1 | ANKK1_HUMAN (Q8NFD2) | ANKK1_MOUSE (Q8BZ25) |
| ARAF | ARAF_HUMAN (P10398) | ARAF_MOUSE (P04627) |
| BMPR1A | BMR1A_HUMAN (P36894) | BMR1A_MOUSE (P36895) |
| BMPR1B | BMR1B_HUMAN (O00238) | BMR1B_MOUSE (P36898) |
| BMPR2 | BMPR2_HUMAN (Q13873) | BMPR2_MOUSE (O35607) |
| BRAF | BRAF_HUMAN (P15056) | BRAF_MOUSE (P28028) |
| ILK | ILK_HUMAN (Q13418) | ILK_MOUSE (O55222) |
| IRAK1 | IRAK1_HUMAN (P51617) | IRAK1_MOUSE (Q62406) |
| IRAK2 | IRAK2_HUMAN (O43187) | IRAK2_MOUSE (Q8CFA1) |
| IRAK3 | IRAK3_HUMAN (Q9Y616) | IRAK3_MOUSE (Q8K4B2) |
| IRAK4 | IRAK4_HUMAN (Q9NWZ3) | IRAK4_MOUSE (Q8R4K2) |
| KSR1 | KSR1_HUMAN (Q8IVT5) | KSR1_MOUSE (Q61097) |
| KSR2 | KSR2_HUMAN (Q6VAB6) | KSR2_MOUSE (Q3UVC0) |
| LIMK1 | LIMK1_HUMAN (P53667) | LIMK1_MOUSE (P53668) |
| LIMK2 | LIMK2_HUMAN (P53671) | LIMK2_MOUSE (O54785) |
| LRRK1 | LRRK1_HUMAN (Q38SD2) | LRRK1_MOUSE (Q3UHC2) |
| LRRK2 | LRRK2_HUMAN (Q5S007) | LRRK2_MOUSE (Q5S006) |
| RAF1 | RAF1_HUMAN (P04049) | RAF1_MOUSE (Q99N57) |
| RIPK1 | RIPK1_HUMAN (Q13546) | RIPK1_MOUSE (Q60855) |
| RIPK2 | RIPK2_HUMAN (O43353) | RIPK2_MOUSE (P58801) |
| RIPK3 | RIPK3_HUMAN (Q9Y572) | RIPK3_MOUSE (Q9QZL0) |
| RIPK4 | RIPK4_HUMAN (P57078) | RIPK4_MOUSE (Q9ERK0) |
| TESK1 | TESK1_HUMAN (Q15569) | TESK1_MOUSE (O70146) |
| TESK2 | TESK2_HUMAN (Q96S53) | TESK2_MOUSE (Q8VCT9) |
| TGFBR1 | TGFR1_HUMAN (P36897) | TGFR1_MOUSE (Q64729) |
| TGFBR2 | TGFR2_HUMAN (P37173) | TGFR2_MOUSE (Q62312) |
| TNNI3K | TNI3K_HUMAN (Q59H18) | TNI3K_MOUSE (Q5GIG6) |

TABLE 1H
Tyr protein kinase family

| | | |
|---|---|---|
| AATK | LMTK1_HUMAN (Q6ZMQ8) | LMTK1_MOUSE (Q80YE4) |
| ABL1 | ABL1_HUMAN (P00519) | ABL1_MOUSE (P00520) |
| ABL2 | ABL2_HUMAN (P42684) | ABL2_MOUSE (Q4JIM5) |
| ALK | ALK_HUMAN (Q9UM73) | ALK_MOUSE (P97793) |
| AXL | UFO_HUMAN (P30530) | UFO_MOUSE (Q00993) |
| BLK | BLK_HUMAN (P51451) | BLK_MOUSE (P16277) |
| BMX | BMX_HUMAN (P51813) | BMX_MOUSE (P97504) |
| BTK | BTK_HUMAN (Q06187) | BTK_MOUSE (P35991) |
| CSF1R | CSF1R_HUMAN (P07333) | CSF1R_MOUSE (P09581) |
| CSK | CSK_HUMAN (P41240) | CSK_MOUSE (P41241) |
| DDR1 | DDR1_HUMAN (Q08345) | DDR1_MOUSE (Q03146) |
| DDR2 | DDR2_HUMAN (Q16832) | DDR2_MOUSE (Q62371) |
| EGFR | EGFR_HUMAN (P00533) | EGFR_MOUSE (Q01279) |
| EPHA1 | EPHA1_HUMAN (P21709) | EPHA1_MOUSE (Q60750) |
| EPHA10 | EPHAA_HUMAN (Q5JZY3) | EPHAA_MOUSE (Q8BYG9) |
| EPHA2 | EPHA2_HUMAN (P29317) | EPHA2_MOUSE (Q03145) |
| EPHA3 | EPHA3_HUMAN (P29320) | EPHA3_MOUSE (P29319) |
| EPHA4 | EPHA4_HUMAN (P54764) | EPHA4_MOUSE (Q03137) |
| EPHA5 | EPHA5_HUMAN (P54756) | EPHA5_MOUSE (Q60629) |
| EPHA6 | EPHA6_HUMAN (Q9UF33) | EPHA6_MOUSE (Q62413) |
| EPHA7 | EPHA7_HUMAN (Q15375) | EPHA7_MOUSE (Q61772) |
| EPHA8 | EPHA8_HUMAN (P29322) | EPHA8_MOUSE (O09127) |
| EPHB1 | EPHB1_HUMAN (P54762) | EPHB1_MOUSE (Q8CBF3) |
| EPHB2 | EPHB2_HUMAN (P29323) | EPHB2_MOUSE (P54763) |
| EPHB3 | EPHB3_HUMAN (P54753) | EPHB3_MOUSE (P54754) |
| EPHB4 | EPHB4_HUMAN (P54760) | EPHB4_MOUSE (P54761) |
| EPHB6 | EPHB6_HUMAN (O15197) | EPHB6_MOUSE (O08644) |
| ERBB2 | ERBB2_HUMAN (P04626) | ERBB2_MOUSE (P70424) |
| ERBB3 | ERBB3_HUMAN (P21860) | ERBB3_MOUSE (Q61526) |
| ERBB4 | ERBB4_HUMAN (Q15303) | ERBB4_MOUSE (Q61527) |
| FER | FER_HUMAN (P16591) | FER_MOUSE (P70451) |
| FES | FES_HUMAN (P07332) | FES_MOUSE (P16879) |
| FGFR1 | FGFR1_HUMAN (P11362) | FGFR1_MOUSE (P16092) |
| FGFR2 | FGFR2_HUMAN (P21802) | FGFR2_MOUSE (P21803) |
| FGFR3 | FGFR3_HUMAN (P22607) | FGFR3_MOUSE (Q61851) |
| FGFR4 | FGFR4_HUMAN (P22455) | FGFR4_MOUSE (Q03142) |
| FGR | FGR_HUMAN (P09769) | FGR_MOUSE (P14234) |
| FLT1 | VGFR1_HUMAN (P17948) | VGFR1_MOUSE (P35969) |
| FLT3 | FLT3_HUMAN (P36888) | FLT3_MOUSE (Q00342) |
| FLT4 | VGFR3_HUMAN (P35916) | VGFR3_MOUSE (P35917) |
| FRK | FRK_HUMAN (P42685) | FRK_MOUSE (Q922K9) |
| FYN | FYN_HUMAN (P06241) | FYN_MOUSE (P39688) |
| HCK | HCK_HUMAN (P08631) | HCK_MOUSE (P08103) |
| IGF1R | IGF1R_HUMAN (P08069) | IGF1R_MOUSE (Q60751) |
| INSR | INSR_HUMAN (P06213) | INSR_MOUSE (P15208) |
| INSRR | INSRR_HUMAN (P14616) | INSRR_MOUSE (Q9WTL4) |
| ITK | ITK_HUMAN (Q08881) | ITK_MOUSE (Q03526) |
| JAK1 | JAK1_HUMAN (P23458) | JAK1_MOUSE (P52332) |
| JAK2 | JAK2_HUMAN (O60674) | JAK2_MOUSE (Q62120) |
| JAK3 | JAK3_HUMAN (P52333) | JAK3_MOUSE (Q62137) |
| KDR | VGFR2_HUMAN (P35968) | VGFR2_MOUSE (P35918) |
| KIT | KIT_HUMAN (P10721) | KIT_MOUSE (P05532) |
| LCK | LCK_HUMAN (P06239) | LCK_MOUSE (P06240) |
| LMTK2 | LMTK2_HUMAN (Q8IWU2) | LMTK2_MOUSE (Q3TYD6) |
| LMTK3 | LMTK3_HUMAN (Q96Q04) | LMTK3_MOUSE (Q5XJV6) |
| LTK | LTK_HUMAN (P29376) | LTK_MOUSE (P08923) |
| LYN | LYN_HUMAN (P07948) | LYN_MOUSE (P25911) |
| MATK | MATK_HUMAN (P42679) | MATK_MOUSE (P41242) |
| MERTK | MERTK_HUMAN (Q12866) | MERTK_MOUSE (Q60805) |
| MET | MET_HUMAN (P08581) | MET_MOUSE (P16056) |
| MST1R | RON_HUMAN (Q04912) | RON_MOUSE (Q62190) |
| MUSK | MUSK_HUMAN (O15146) | MUSK_MOUSE (Q61006) |
| NTRK1 | NTRK1_HUMAN (P04629) | NTRK1_MOUSE (Q3UFB7) |
| NTRK2 | NTRK2_HUMAN (Q16620) | NTRK2_MOUSE (P15209) |
| NTRK3 | NTRK3_HUMAN (Q16288) | NTRK3_MOUSE (Q6VNS1) |
| PDGFRA | PGFRA_HUMAN (P16234) | PGFRA_MOUSE (P26618) |
| PDGFRB | PGFRB_HUMAN (P09619) | PGFRB_MOUSE (P05622) |
| PTK2 | FAK1_HUMAN (Q05397) | FAK1_MOUSE (P34152) |
| PTK2B | FAK2_HUMAN (Q14289) | FAK2_MOUSE (Q9QVP9) |
| PTK6 | PTK6_HUMAN (Q13882) | PTK6_MOUSE (Q64434) |
| PTK7 | PTK7_HUMAN (Q13308) | PTK7_MOUSE (Q8BKG3) |
| RET | RET_HUMAN (P07949) | RET_MOUSE (P35546) |
| ROR1 | ROR1_HUMAN (Q01973) | ROR1_MOUSE (Q9Z139) |
| ROR2 | ROR2_HUMAN (Q01974) | ROR2_MOUSE (Q9Z138) |
| ROS1 | ROS1_HUMAN (P08922) | ROS1_MOUSE (Q78DX7) |
| RYK | RYK_HUMAN (P34925) | RYK_MOUSE (Q01887) |
| SRC | SRC_HUMAN (P12931) | SRC_MOUSE (P05480) |
| SRMS | SRMS_HUMAN (Q9H3Y6) | SRMS_MOUSE (Q62270) |
| STYK1 | STYK1_HUMAN (Q6J9G0) | STYK1_MOUSE (Q6J9G1) |
| SYK | KSYK_HUMAN (P43405) | KSYK_MOUSE (P48025) |
| Smok1 | | SMOK1_MOUSE (Q9QYZ4) |
| Smoktcr | | SMKTR_MOUSE (A2KF29) |
| TEC | TEC_HUMAN (P42680) | TEC_MOUSE (P24604) |
| TEK | TIE2_HUMAN (Q02763) | TIE2_MOUSE (Q02858) |
| TIE1 | TIE1_HUMAN (P35590) | TIE1_MOUSE (Q06806) |
| TNK1 | TNK1_HUMAN (Q13470) | TNK1_MOUSE (Q99ML2) |
| TNK2 | ACK1_HUMAN (Q07912) | ACK1_MOUSE (O54967) |
| TXK | TXK_HUMAN (P42681) | TXK_MOUSE (P42682) |
| TYK2 | TYK2_HUMAN (P29597) | TYK2_MOUSE (Q9R117) |
| TYRO3 | TYRO3_HUMAN (Q06418) | TYRO3_MOUSE (P55144) |
| YES1 | YES_HUMAN (P07947) | YES_MOUSE (Q04736) |
| ZAP70 | ZAP70_HUMAN (P43403) | ZAP70_MOUSE (P43404) |

TABLE 1I
Other kinases.

| | | |
|---|---|---|
| AAK1 | AAK1_HUMAN (Q2M2I8) | AAK1_MOUSE (Q3UHJ0) |
| AURKA | AURKA_HUMAN (O14965) | AURKA_MOUSE (P97477) |
| AURKB | AURKB_HUMAN (Q96GD4) | AURKB_MOUSE (O70126) |
| AURKC | AURKC_HUMAN (Q9UQB9) | AURKC_MOUSE (O88445) |
| BMP2K | BMP2K_HUMAN (Q9NSY1) | BMP2K_MOUSE (Q91Z96) |
| BUB1 | BUB1_HUMAN (O43683) | BUB1_MOUSE (O08901) |
| BUB1B | BUB1B_HUMAN (O60566) | BUB1B_MOUSE (Q9Z1S0) |
| CAMKK1 | KKCC1_HUMAN (Q8N559) | KKCC1_MOUSE (Q8VBY2) |
| CAMKK2 | KKCC2_HUMAN (Q96RR4) | KKCC2_MOUSE (Q8C078) |
| CDC7 | CDC7_HUMAN (O00311) | CDC7_MOUSE (Q9Z0H0) |
| CHUK | IKKA_HUMAN (O15111) | IKKA_MOUSE (Q60680) |
| CSNK2A1 | CSK21_HUMAN (P68400) | CSK21_MOUSE (Q60737) |
| CSNK2A2 | CSK22_HUMAN (P19784) | CSK22_MOUSE (O54833) |
| CSNK2A3 | CSK23_HUMAN (Q8NEV1) | |
| DSTYK | DUSTY_HUMAN (Q6XUX3) | DUSTY_MOUSE (Q6XUX1) |
| EIF2AK1 | E2AK1_HUMAN (Q9BQI3) | E2AK1_MOUSE (Q9Z2R9) |
| EIF2AK2 | E2AK2_HUMAN (P19525) | E2AK2_MOUSE (Q03963) |
| EIF2AK3 | E2AK3_HUMAN (Q9NZJ5) | E2AK3_MOUSE (Q9Z2B5) |
| EIF2AK4 | E2AK4_HUMAN (Q9P2K8) | E2AK4_MOUSE (Q9QZ05) |
| ERN1 | ERN1_HUMAN (O75460) | ERN1_MOUSE (Q9EQY0) |
| ERN2 | ERN2_HUMAN (Q76MJ5) | ERN2_MOUSE (Q9Z2E3) |
| GAK | GAK_HUMAN (O14976) | GAK_MOUSE (Q99KY4) |
| HASPIN | HASP_HUMAN (Q8TF76) | HASP_MOUSE (Q9Z0R0) |
| IKBKB | IKKB_HUMAN (O14920) | IKKB_MOUSE (O88351) |
| IKBKE | IKKE_HUMAN (Q14164) | IKKE_MOUSE (Q9R0T8) |
| MLKL | MLKL_HUMAN (Q8NB16) | MLKL_MOUSE (Q9D2Y4) |
| MOS | MOS_HUMAN (P00540) | MOS_MOUSE (P00536) |
| NRBP1 | NRBP_HUMAN (Q9UHY1) | NRBP_MOUSE (Q99J45) |
| NRBP2 | NRBP2_HUMAN (Q9NSY0) | NRBP2_MOUSE (Q91V36) |
| PAN3 | PAN3_HUMAN (Q58A45) | PAN3_MOUSE (Q640Q5) |
| PDIK1L | PDK1L_HUMAN (Q8N165) | PDK1L_MOUSE (Q8QZR7) |
| PEAK1 | PEAK1_HUMAN (Q9H792) | PEAK1_MOUSE (Q69Z38) |
| PIK3R4 | PI3R4_HUMAN (Q99570) | PI3R4_MOUSE (Q8VD65) |
| PINK1 | PINK1_HUMAN (Q9BXM7) | PINK1_MOUSE (Q99MQ3) |
| PKDCC | PKDCC_HUMAN (Q504Y2) | PKDCC_MOUSE (Q5RJI4) |
| PKMYT1 | PMYT1_HUMAN (Q99640) | PMYT1_MOUSE (Q9ESG9) |
| PLK1 | PLK1_HUMAN (P53350) | PLK1_MOUSE (Q07832) |
| PLK2 | PLK2_HUMAN (Q9NYY3) | PLK2_MOUSE (P53351) |
| PLK3 | PLK3_HUMAN (Q9H4B4) | PLK3_MOUSE (Q60806) |
| PLK4 | PLK4_HUMAN (O00444) | PLK4_MOUSE (Q64702) |
| PLK5 | PLK5_HUMAN (Q496M5) | PLK5_MOUSE (Q4FZD7) |
| POMK | SG196_HUMAN (Q9H5K3) | SG196_MOUSE (Q3TUA9) |
| PRAG1 | PRAG1_HUMAN (Q86YV5) | PRAG1_MOUSE (Q571I4) |
| PXK | PXK_HUMAN (Q7Z7A4) | PXK_MOUSE (Q8BX57) |
| RNASEL | RN5A_HUMAN (Q05823) | RN5A_MOUSE (Q05921) |
| RPS6KC1 | KS6C1_HUMAN (Q96S38) | KS6C1_MOUSE (Q8BLK9) |
| RPS6KL1 | RPKL1_HUMAN (Q9Y6S9) | RPKL1_MOUSE (Q8R2S1) |
| SBK1 | SBK1_HUMAN (Q52WX2) | SBK1_MOUSE (Q8QZX0) |
| SBK2 | SBK2_HUMAN (P0C263) | SBK2_MOUSE (P0C5K1) |
| SBK3 | SBK3_HUMAN (P0C264) | SBK3_MOUSE (P0C5K0) |
| SCYL1 | SCYL1_HUMAN (Q96KG9) | SCYL1_MOUSE (Q9EQC5) |
| SCYL2 | SCYL2_HUMAN (Q6P3W7) | SCYL2_MOUSE (Q8CFE4) |
| SCYL3 | PACE1_HUMAN (Q8IZE3) | PACE1_MOUSE (Q9DBQ7) |
| SGK494 | SG494_HUMAN (Q96LW2) | SG494_MOUSE (Q5SYL1) |
| STK16 | STK16_HUMAN (O75716) | STK16_MOUSE (O88697) |
| STK31 | STK31_HUMAN (Q9BXU1) | STK31_MOUSE (Q99MW1) |
| STK32A | ST32A_HUMAN (Q8WU08) | ST32A_MOUSE (Q8BGW6) |
| STK32B | ST32B_HUMAN (Q9NY57) | ST32B_MOUSE (Q9JJX8) |
| STK32C | ST32C_HUMAN (Q86UX6) | ST32C_MOUSE (Q8QZV4) |
| STK35 | STK35_HUMAN (Q8TDR2) | STK35_MOUSE (Q80ZW0) |
| STK36 | STK36_HUMAN (Q9NRP7) | STK36_MOUSE (Q69ZM6) |
| STKLD1 | STKL1_HUMAN (Q8NE28) | STKL1_MOUSE (Q80YS9) |
| TBCK | TBCK_HUMAN (Q8TEA7) | TBCK_MOUSE (Q8BM85) |
| TBK1 | TBK1_HUMAN (Q9UHD2) | TBK1_MOUSE (Q9WUN2) |
| TEX14 | TEX14_HUMAN (Q8IWB6) | TEX14_MOUSE (Q7M6U3) |
| TLK1 | TLK1_HUMAN (Q9UKI8) | TLK1_MOUSE (Q8C0V0) |
| TLK2 | TLK2_HUMAN (Q86UE8) | TLK2_MOUSE (O55047) |
| TP53RK | PRPK_HUMAN (Q96S44) | PRPK_MOUSE (Q99PW4) |
| TTK | TTK_HUMAN (P33981) | TTK_MOUSE (P35761) |
| UHMK1 | UHMK1_HUMAN (Q8TAS1) | UHMK1_MOUSE (P97343) |
| ULK1 | ULK1_HUMAN (O75385) | ULK1_MOUSE (O70405) |
| ULK2 | ULK2_HUMAN (Q8IYT8) | ULK2_MOUSE (Q9QY01) |
| ULK3 | ULK3_HUMAN (Q6PHR2) | ULK3_MOUSE (Q3U3Q1) |
| ULK4 | ULK4_HUMAN (Q96C45) | ULK4_MOUSE (Q3V129) |
| WEE1 | WEE1_HUMAN (P30291) | WEE1_MOUSE (P47810) |
| WEE2 | WEE2_HUMAN (P0C1S8) | WEE2_MOUSE (Q66JT0) |
| WNK1 | WNK1_HUMAN (Q9H4A3) | WNK1_MOUSE (P83741) |
| WNK2 | WNK2_HUMAN (Q9Y3S1) | WNK2_MOUSE (Q3UH66) |
| WNK3 | WNK3_HUMAN (Q9BYP7) | WNK3_MOUSE (Q80XP9) |
| WNK4 | WNK4_HUMAN (Q96J92) | WNK4_MOUSE (Q80UE6) |

TABLE 1J
ADCK protein kinase family

| | | |
|---|---|---|
| ADCK1 | ADCK1_HUMAN (Q86TW2) | ADCK1_MOUSE (Q9D0L4) |
| ADCK2 | ADCK2_HUMAN (Q7Z695) | ADCK2_MOUSE (Q6NSR3) |
| ADCK5 | ADCK5_HUMAN (Q3MIX3) | ADCK5_MOUSE (Q80V03) |
| COQ8A | COQ8A_HUMAN (Q8NI60) | COQ8A_MOUSE (Q60936) |
| COQ8B | COQ8B_HUMAN (Q96D53) | COQ8B_MOUSE (Q566J8) |

TABLE 1K
Alpha-type protein kinase family

| | | |
|---|---|---|
| ALPK1 | ALPK1_HUMAN (Q96QP1) | ALPK1_MOUSE (Q9CXB8) |
| ALPK2 | ALPK2_HUMAN (Q86TB3) | ALPK2_MOUSE (Q91ZB0) |
| ALPK3 | ALPK3_HUMAN (Q96L96) | ALPK3_MOUSE (Q924C5) |
| EEF2K | EF2K_HUMAN (O00418) | EF2K_MOUSE (O08796) |
| TRPM6 | TRPM6_HUMAN (Q9BX84) | TRPM6_MOUSE (Q8CIR4) |
| TRPM7 | TRPM7_HUMAN (Q96QT4) | TRPM7_MOUSE (Q923J1) |

TABLE 1L
FAST protein kinase family

| | | |
|---|---|---|
| FASTK | FASTK_HUMAN (Q14296) | FASTK_MOUSE (Q9JIX9) |

TABLE 1M
PDK/BCKDK protein kinase family

| | | |
|---|---|---|
| BCKDK | BCKD_HUMAN (O14874) | BCKD_MOUSE (O55028) |
| PDK1 | PDK1_HUMAN (Q15118) | PDK1_MOUSE (Q8BFP9) |
| PDK2 | PDK2_HUMAN (Q15119) | PDK2_MOUSE (Q9JK42) |
| PDK3 | PDK3_HUMAN (Q15120) | PDK3_MOUSE (Q922H2) |
| PDK4 | PDK4_HUMAN (Q16654) | PDK4_MOUSE (O70571) |

TABLE 1N
PI3/PI4-kinase family

| | | |
|---|---|---|
| ATM | ATM_HUMAN (Q13315) | ATM_MOUSE (Q62388) |
| ATR | ATR_HUMAN (Q13535) | ATR_MOUSE (Q9JKK8) |
| MTOR | MTOR_HUMAN (P42345) | MTOR_MOUSE (Q9JLN9) |
| PIK3CA | PK3CA_HUMAN (P42336) | PK3CA_MOUSE (P42337) |
| PIK3CG | PK3CG_HUMAN (P48736) | PK3CG_MOUSE (Q9JHG7) |
| PRKDC | PRKDC_HUMAN (P78527) | PRKDC_MOUSE (P97313) |
| SMG1 | SMG1_HUMAN (Q96Q15) | SMG1_MOUSE (Q8BKX6) |

TABLE 1O
RIO-type Ser/Thr kinase family

| | | |
|---|---|---|
| RIOK1 | RIOK1_HUMAN (Q9BRS2) | RIOK1_MOUSE (Q922Q2) |
| RIOK2 | RIOK2_HUMAN (Q9BVS4) | RIOK2_MOUSE (Q9CQS5) |
| RIOK3 | RIOK3_HUMAN (O14730) | RIOK3_MOUSE (Q9DBU3) |

### IV. Systems and Methods

In some embodiments, provided herein are systems and methods to enhance engagement (e.g., binding) of a kinase target with the kinase binding moiety of a functional kinase binding agent using an active variant of KRAS (e.g., KRAS4A variant, KRAS4B variant, etc.). In some embodiments, engagement of the kinase by the kinase binding moiety of a functional kinase binding agent allows for detection, isolation, analyzing, quantification, characterization, etc. of kinases within a sample (e.g., a cell, a cell lysate, a sample, a biochemical solution or mixture, a tissue, an organism, etc.). In some embodiments, an active variant of KRAS (e.g., KRAS4A variant, KRAS4B variant, etc.) is added to the sample or system. In some embodiments, an active variant of KRAS (e.g., KRAS4A variant, KRAS4B variant, etc.) is expressed within the sample or system.

In some embodiments, provided herein are methods of detecting one or more kinases in a sample, the method comprising contacting the sample with a functional kinase binding agent in the presence of an active variant of KRAS (e.g., KRAS4A variant, KRAS4B variant, etc.). In some embodiments, provided herein are methods to isolate one or more kinases from a sample.

In some embodiments, methods are provided for characterizing a sample by analyzing the presence, quantity, and or population of kinases in the sample (e.g., what kinases are present and/or at what quantities) in the presence of an active variant of KRAS (e.g., KRAS4A variant, KRAS4B variant, etc.) by contacting the sample with a functional kinase binding agent.

In some embodiments, kinases bound by functional kinase binding agents are detected, quantified, and/or isolated by taking advantage of unique properties of the functional element by any means including electrophoresis, gel filtration, high-pressure or fast-pressure liquid chromatography, mass spectroscopy, affinity chromatography, ion exchange chromatography, chemical extraction, magnetic bead separation, precipitation, hydrophobic interaction chromatography (HIC), or any combination thereof. The isolated kinase(s) may be employed for structural and functional studies, for diagnostic applications, for the preparation biological or pharmaceutical reagents, as a tool for the development of drugs, and for studying protein interactions, for the isolation and characterization of protein complexes, etc.

In some embodiments, methods are provided for detecting and/or quantifying a functional kinase binding agent and/or a kinase or protein complex (e.g., comprising a kinase) bound thereto in a sample comprising an active variant of KRAS (e.g., KRAS4A variant, KRAS4B variant, etc.). In some embodiments, techniques for detection and/or quantification of the functional kinase binding agents and/or analytes (e.g., kinases) bound thereto depend upon the identity of the detectable element of the functional kinase binding agent (e.g., fluorophore, luciferase, chelated radionuclide, chelated contrast agent, etc.) and/or specific modifications to the functional kinase binding agent (e.g., mass tags (e.g., heavy isotopes (e.g., ¹³C, ¹⁵N, ²H, etc.). For example, when a functional kinase binding agent herein comprises a fluorophore or other light emitting detectable element, the compound and/or analyte (e.g., kinases) bound thereto may be detected/quantified in a sample using systems, devices, and/or apparatuses that are provided to detect, quantitate, or monitor, the amount of light (e.g., fluorescence) emitted, or changes thereto. In some embodiments, detection, quantification, and/or monitoring are provided by a device, system or apparatus comprising one or more of a spectrophotometer, fluorometer, luminometer, photomultiplier tube, photodiode, nephlometer, photon counter, electrodes, ammeter, voltmeter, capacitative sensors, flow cytometer, CCD, etc.

In addition to fluorescent detectable elements, functional kinase binding agents may comprise a variety of detectable elements with physical properties based on the interaction and response of the detectable elements to electromagnetic fields and radiation, which can be used to detect the tracers and/or a bound kinase. These properties include absorption in the UV, visible, and infrared regions of the electromagnetic spectrum, presence of chromophores that are Raman active and can be further enhanced by resonance Raman spectroscopy, electron spin resonance activity and nuclear magnetic resonances and molecular mass, e.g., via a mass spectrometer.

In some embodiments, systems are provided comprising: (a) a fusion of a protein kinase (e.g., of Table 1A-O or a variant thereof) and a bioluminescent protein; (b) an active variant of KRAS (e.g., KRAS4A variant, KRAS4B variant, etc.); and (c) a functional kinase binding agent comprising a kinase binding moiety and an energy acceptor (e.g., fluorophore); wherein the emission spectrum of the bioluminescent protein overlaps the excitation spectrum of the energy acceptor (e.g., fluorophore), such that BRET is detectable between the bioluminescent protein and the energy acceptor (e.g., fluorophore) when the kinase binding moiety binds to the protein kinase. Similar BRET systems (e.g., utilizing a NANOLUC® luciferase) are described in, for example, Intl. Pat. App. PCT/US13/74765 (herein incorporated by reference in its entirety); embodiments of which will find use in the systems and methods herein.

### EXPERIMENTAL

### Example 1

### Enhancement of Intracellular Target Engagement by Through Co-Expression of KRAS4B^{G12C}

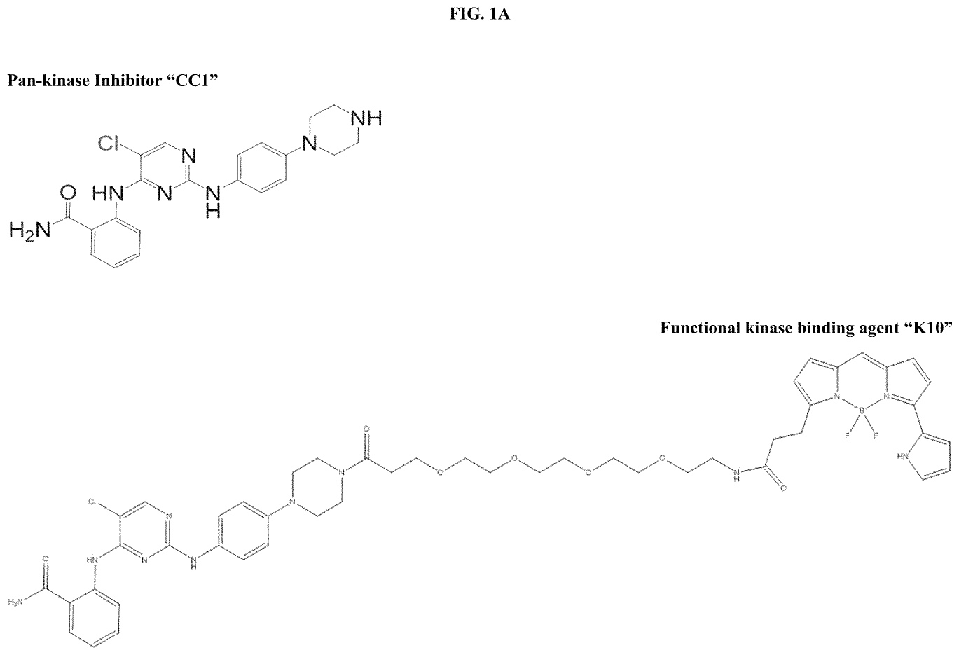

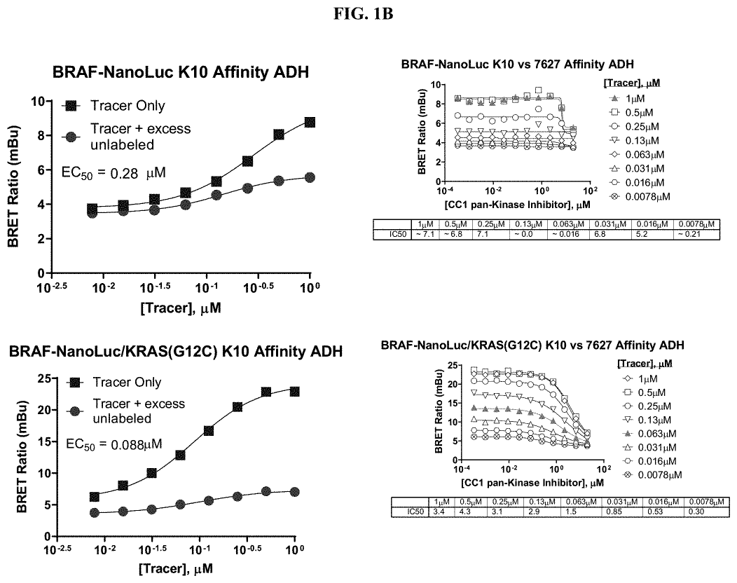

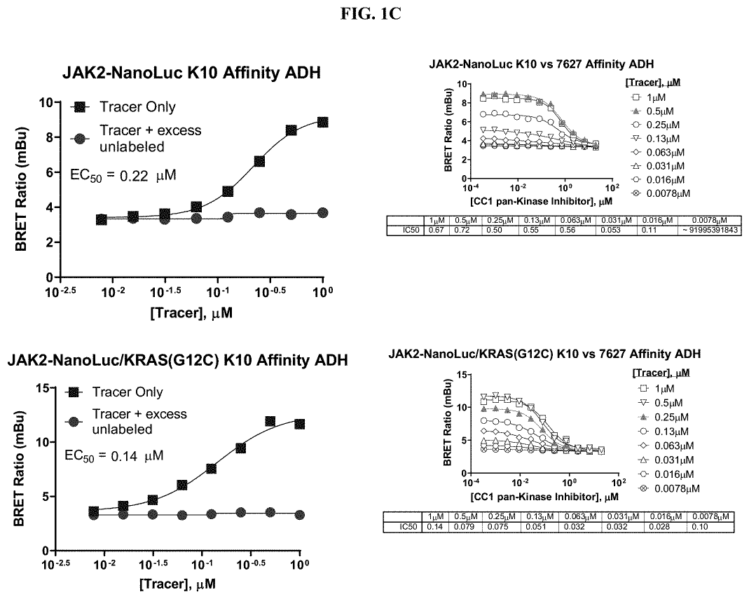

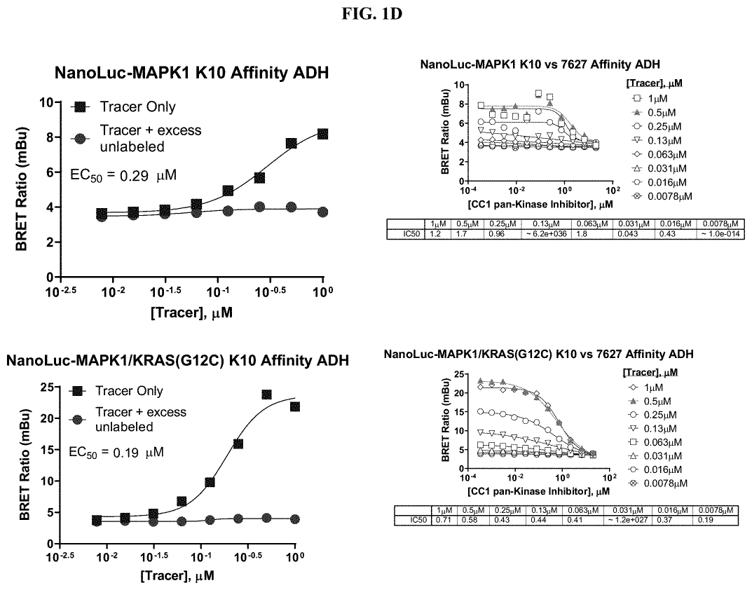

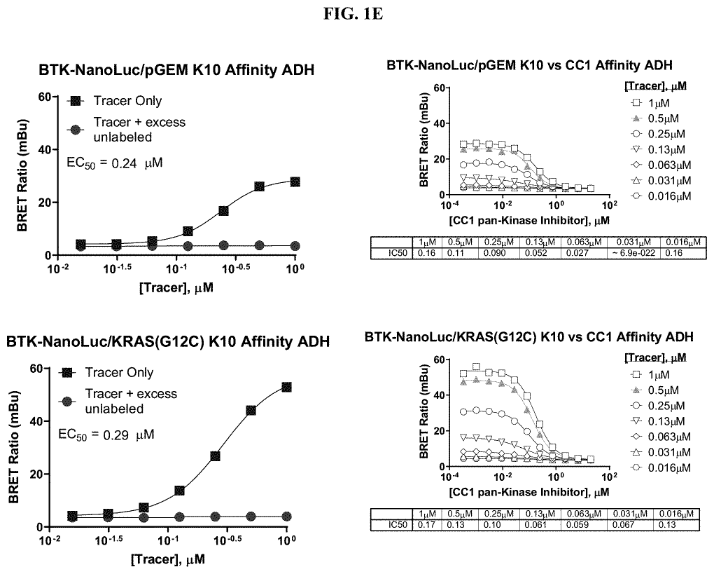

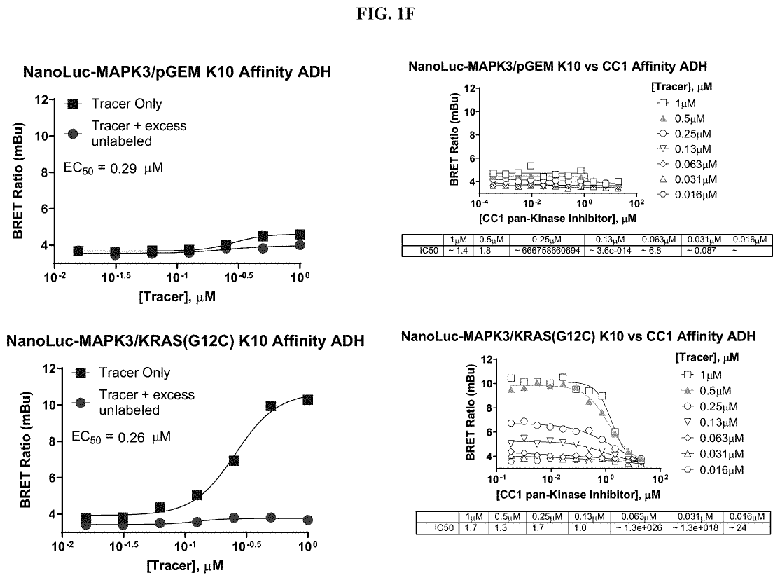

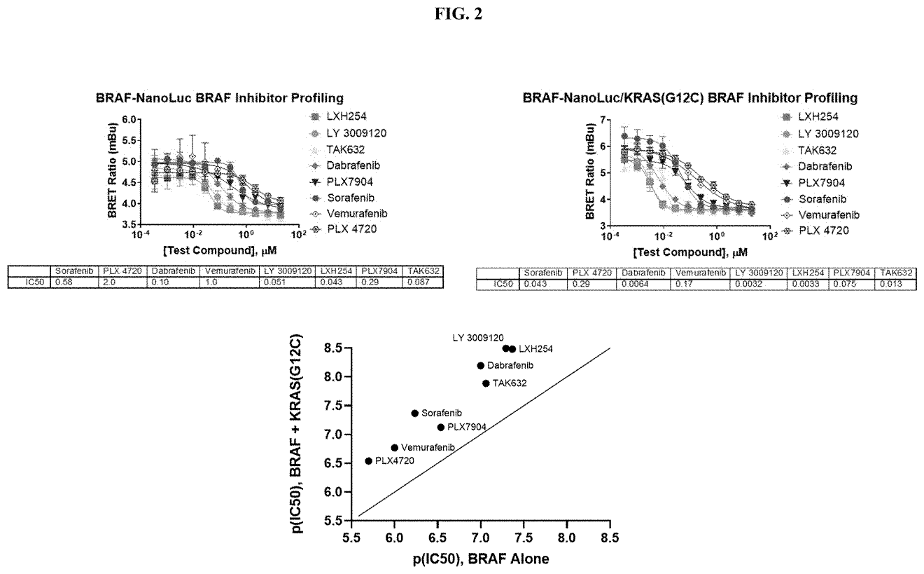

Experiments were conducted during development of embodiments herein to demonstrate enhanced NanoBRET live cell target engagement via co-expression of KRAS4B^{G12C}. In wells of 96-well plates, 20,000 HEK293 cells per well were transfected with kinase/NanoLuc (Nuc) fusions expressed from pFN31K and pFN32K plasmids. Transfections were performed using 3:1 FuGENE HD:plasmid ratios. Each kinase/Nluc fusion was co-transfected with a pF5 vector encoding untagged KRAS4B^{G12C} or transfection carrier DNA/pGEM (1 part kinase/Nluc:9 parts KRAS^{G12C} or transfection carrier DNA/pGEM). 24 hours post transfection, cells were treated for 2 hours in the presence of Tracer K10 and varying concentrations of control inhibitor CC1 (FIG. 1A). After incubation, NanoBRET-TE substrate/inhibitor solution (Promega Corporation) was added to a final concentration of 1×, and BRET was measured on a Glomax® Discover plate reader (FIG. 1B-F). Co-expression of KRAS^{G12C} potentiated the NanoBRET signal with the tracer. Enhanced target engagement of unmodified drug was also observed. KRAS4B^{G12C} co-expression enhanced target engagement for all of the targets in this analysis.

### Example 2

### Understanding the Role of KRAS in Activating Kinase Signaling Pathway in Cancer Cells

KRAS, and related cell signaling pathways, are among the most important therapeutic targets in oncology. However, beyond the MAPK pathway, the cell signaling events modulated by mutant KRAS activity are not completely elucidated. Therefore, methods to determine the cellular processes and novel oncogenic pathways influenced KRAS activity are critical for ongoing drug discovery efforts.

In cells, expression of an active KRAS variant may result in activation of a signal transduction pathway or other cellular process. Activation of signal transduction pathways generally increases kinase post-translational modifications events (e.g., phosphorylation). Commonly, altered kinase phosphorylation is commensurate with enhanced target engagement potency. Therefore, activation of KRAS signaling pathways may cause a change in kinase post-translational modifications commensurate with enhanced kinase target engagement. Increases in kinase target engagement could therefore serve as a detectable signal to elucidate novel KRAS-related cellular processes. It is contemplated that a method relying on changes in such signals is capable of uncovering novel targets for therapeutic intervention and drug development.

SEQUENCES
KRAS4A (nucleotide sequence)
SEQ ID NO: 1
atgactgaatataaacttgtggtagttggagctggtggcgtaggcaagagtgccttgacgatacagct
aattcagaatcattttgtggacgaatatgatccaacaatagaggattcctacaggaagcaagtagtaa
ttgatggagaaacctgtctcttggatattctcgacacagcaggtcaagaggagtacagtgcaatgagg
gaccagtacatgaggactggggagggctttctttgtgtatttgccataaataatactaaatcatttga
agatattcaccattatagagaacaaattaaaagagttaaggactctgaagatgtacctatggtcctag
taggaaataaatgtgatttgccttctagaacagtagacacaaaacaggctcaggacttagcaagaagt
tatggaattccttttattgaaacatcagcaaagacaagacagagagtggaggatgctttttatacatt
ggtgagagagatccgacaatacagattgaaaaaaatcagcaaagaagaaaagactcctggctgtgtga
aaattaaaaaatgcattataatg
KRAS4A (protein sequence)
SEQ ID NO: 2
MTEYKLVVVGAGGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4A^{G34T} (nucleotide sequence)
SEQ ID NO: 3
atgactgaatataaacttgtggtagttggagcttgtggcgtaggcaagagtgccttgacgatacagct
aattcagaatcattttgtggacgaatatgatccaacaatagaggattcctacaggaagcaagtagtaa
ttgatggagaaacctgtctcttggatattctcgacacagcaggtcaagaggagtacagtgcaatgagg
gaccagtacatgaggactggggagggctttctttgtgtatttgccataaataatactaaatcatttga
agatattcaccattatagagaacaaattaaaagagttaaggactctgaagatgtacctatggtcctag
taggaaataaatgtgatttgccttctagaacagtagacacaaaacaggctcaggacttagcaagaagt
tatggaattccttttattgaaacatcagcaaagacaagacagagagtggaggatgctttttatacatt
ggtgagagagatccgacaatacagattgaaaaaaatcagcaaagaagaaaagactcctggctgtgtga
aaattaaaaaatgcattataatg
KRAS4A^{G12C} (protein sequence)
SEQ ID NO: 4
MTEYKLVVVGACGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4A^{G35A} (nucleotide sequence)
SEQ ID NO: 5
atgactgaatataaacttgtggtagttggagctgatggcgtaggcaagagtgccttgacgatacagct
aattcagaatcattttgtggacgaatatgatccaacaatagaggattcctacaggaagcaagtagtaa
ttgatggagaaacctgtctcttggatattctcgacacagcaggtcaagaggagtacagtgcaatgagg
gaccagtacatgaggactggggagggctttctttgtgtatttgccataaataatactaaatcatttga
agatattcaccattatagagaacaaattaaaagagttaaggactctgaagatgtacctatggtcctag
taggaaataaatgtgatttgccttctagaacagtagacacaaaacaggctcaggacttagcaagaagt
tatggaattccttttattgaaacatcagcaaagacaagacagagagtggaggatgctttttatacatt
ggtgagagagatccgacaatacagattgaaaaaaatcagcaaagaagaaaagactcctggctgtgtga
aaattaaaaaatgcattataatg
KRAS4A^{G12D} (protein sequence)
SEQ ID NO: 6
MTEYKLVVVGADGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4A^{G35T} (nucleotide sequence)
SEQ ID NO: 7
atgactgaatataaacttgtggtagttggagctgttggcgtaggcaagagtgccttgacgatacagct
aattcagaatcattttgtggacgaatatgatccaacaatagaggattcctacaggaagcaagtagtaa
ttgatggagaaacctgtctcttggatattctcgacacagcaggtcaagaggagtacagtgcaatgagg
gaccagtacatgaggactggggagggctttctttgtgtatttgccataaataatactaaatcatttga
agatattcaccattatagagaacaaattaaaagagttaaggactctgaagatgtacctatggtcctag
taggaaataaatgtgatttgccttctagaacagtagacacaaaacaggctcaggacttagcaagaagt
tatggaattccttttattgaaacatcagcaaagacaagacagagagtggaggatgctttttatacatt
ggtgagagagatccgacaatacagattgaaaaaaatcagcaaagaagaaaagactcctggctgtgtga
aaattaaaaaatgcattataatg
KRAS4A^{G12V} (protein sequence)
SEQ ID NO: 8
MTEYKLVVVGAVGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQRVEDAFYTLVREIRQYRLKKISKEEKTPGCVKIKKCIIM
KRAS4B (nucleotide sequence)
SEQ ID NO: 9
atgactgaatataaacttgtggtagttggagctggtggcgtaggcaagagcaacaatagaggattcct
acaggaagcaagtagtaattgatggagaaacctgtctcttggatattctcgacacagcaggtcaagag
gagtacagtgcaatgagggaccagtacatgaggactggggagggctttctttgtgtatttgccataaa
taatactaaatcatttgaagatattcaccattatagagaacaaattaaaagagttaaggactctgaag
atgtacctatggtcctagtaggaaataaatgtgatttgccttctagaacagtagacacaaaacaggct
caggacttagcaagaagttatggaattccttttattgaaacatcagcaaagacaagacagggtgttga
tgatgccttctatacattagttcgagaaattcgaaaacataaagaaaagatgagcaaagatggtaaaa
agaagaaaaagaagtcaaagacaaagtgtgtaattatgtaa
KRAS4B (protein sequence)
SEQ ID NO: 10
MTEYKLVVVGAGGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
KRAS4B^{G34T} (nucleotide sequence)
SEQ ID NO: 11
atgactgaatataaacttgtggtagttggagcttgtggcgtaggcaagagcaacaatagaggattcct
acaggaagcaagtagtaattgatggagaaacctgtctcttggatattctcgacacagcaggtcaagag
gagtacagtgcaatgagggaccagtacatgaggactggggagggctttctttgtgtatttgccataaa
taatactaaatcatttgaagatattcaccattatagagaacaaattaaaagagttaaggactctgaag
atgtacctatggtcctagtaggaaataaatgtgatttgccttctagaacagtagacacaaaacaggct
caggacttagcaagaagttatggaattccttttattgaaacatcagcaaagacaagacagggtgttga
tgatgccttctatacattagttcgagaaattcgaaaacataaagaaaagatgagcaaagatggtaaaa
agaagaaaaagaagtcaaagacaaagtgtgtaattatgtaa
KRAS4B^{G12C} (protein sequence)
SEQ ID NO: 12
MTEYKLVVVGACGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
KRAS4B^{G35A} (nucleotide sequence)
SEQ ID NO: 13
atgactgaatataaacttgtggtagttggagctgatggcgtaggcaagagcaacaatagaggattcct
acaggaagcaagtagtaattgatggagaaacctgtctcttggatattctcgacacagcaggtcaagag
gagtacagtgcaatgagggaccagtacatgaggactggggagggctttctttgtgtatttgccataaa
taatactaaatcatttgaagatattcaccattatagagaacaaattaaaagagttaaggactctgaag
atgtacctatggtcctagtaggaaataaatgtgatttgccttctagaacagtagacacaaaacaggct
caggacttagcaagaagttatggaattccttttattgaaacatcagcaaagacaagacagggtgttga
tgatgccttctatacattagttcgagaaattcgaaaacataaagaaaagatgagcaaagatggtaaaa
agaagaaaaagaagtcaaagacaaagtgtgtaattatgtaa
KRAS4B^{G12D} (protein sequence)
SEQ ID NO: 14
MTEYKLVVVGADGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
KRAS4B^{G35T} (nucleotide sequence)
SEQ ID NO: 15
atgactgaatataaacttgtggtagttggagctgttggcgtaggcaagagcaacaatagaggattcct
acaggaagcaagtagtaattgatggagaaacctgtctcttggatattctcgacacagcaggtcaagag
gagtacagtgcaatgagggaccagtacatgaggactggggagggctttctttgtgtatttgccataaa
taatactaaatcatttgaagatattcaccattatagagaacaaattaaaagagttaaggactctgaag
atgtacctatggtcctagtaggaaataaatgtgatttgccttctagaacagtagacacaaaacaggct
caggacttagcaagaagttatggaattccttttattgaaacatcagcaaagacaagacagggtgttga
tgatgccttctatacattagttcgagaaattcgaaaacataaagaaaagatgagcaaagatggtaaaa
agaagaaaaagaagtcaaagacaaagtgtgtaattatgtaa
KRAS4B^{G12V} (protein sequence)
SEQ ID NO: 16
MTEYKLVVVGAVGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM
NanoLuc (nucleotide sequence)
SEQ ID NO: 17
atgaaacatcaccatcaccatcatgcgatcgccatggtcttcacactcgaagatttcgttggggactg
gcgacagacagccggctacaacctggaccaagtccttgaacagggaggtgtgtccagtttgtttcaga
atctcggggtgtccgtaactccgatccaaaggattgtcctgagcggtgaaaatgggctgaagatcgac
atccatgtcatcatcccgtatgaaggtctgagcggcgaccaaatgggccagatcgaaaaaatttttaa
ggtggtgtaccctgtggatgatcatcactttaaggtgatcctgcactatggcacactggtaatcgacg
gggttacgccgaacatgatcgactatttcggacggccgtatgaaggcatcgccgtgttcgacggcaaa
aagatcactgtaacagggaccctgtggaacggcaacaaaattatcgacgagcgcctgatcaaccccga
cggctccctgctgttccgagtaaccatcaacggagtgaccggctggcggctgtgcgaacgcattctgg
cggtt
NanoLuc (protein sequence)
SEQ ID NO: 18
MKHHHHHHAIAMVFTLEDFVGDWRQTAGYNLDQVLEQGGVSSLFQNLGVSVTPIQRIVLSGENGLKID
IHVIIPYEGLSGDQMGQIEKIFKVVYPVDDHHFKVILHYGTLVIDGVTPNMIDYFGRPYEGIAVFDGK
KITVTGTLWNGNKIIDERLINPDGSLLFRVTINGVTGWRLCERILAV

1. A method of detecting or quantifying a kinase in a sample, comprising:
(a) providing a sample comprising the kinase and an active KRAS variant; and
(b) contacting the sample with a kinase binding agent comprising a functional element.

2. The method of claim 1, wherein the kinase binding agent is a functionalized kinase binding agent and comprises a kinase binding moiety and a functional element.

3. The method of claim 2, further comprising (c) detecting or quantifying the functional element

4. The method of claim 1, wherein the kinase binding agent consists of a kinase binding moiety.

5. The method of claim 1, wherein step (a) comprises contacting a sample comprising the kinase with the active KRAS variant.

6. The method of claim 1, wherein step (a) comprises expressing the kinase and the active KRAS variant within the sample.

7. The method of claim 1, wherein the active KRAS variant is an active variant of KRAS4A.

8. The method of claim 7, wherein the active variant of KRAS4A is KRAS4A^{G12C}.

9. The method of claim 1, wherein the active KRAS variant is an active variant of KRAS4B.

10. The method of claim 1, wherein the active KRAS variant is an active variant comprises a substitution at position 12.

11. The method of claim 2, wherein the functional element is a detectable element, an affinity element, a capture element, or a solid support.

12. The method of claim 11, wherein the functional element is a detectable element selected from a fluorophore, chromophore, radionuclide, electron opaque molecule, an Mill contrast agent, SPECT contrast agent, and mass tag.

13. The method of claim 11, wherein the detectable element or the signal produced thereby is detected or quantified by fluorescence, mass spectrometry, optical imaging, magnetic resonance imaging (MM), or energy transfer.

14. The method of claim 11, wherein the functional element is a solid support selected from a sedimental particle, a membrane, glass, a tube, a well, a self-assembled monolayer, a surface plasmon resonance chip, and a solid support with an electron conducting surface.

15. The method of claim 14, wherein the sedimental particle is a magnetic particle.

16. The method of claim 2, wherein the functional kinase binding agent is of the formula: 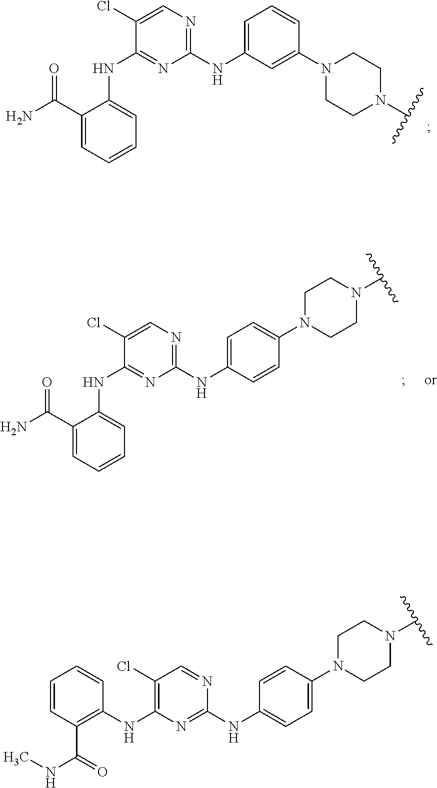 [SMILES: *N1CCN(c2cccc(Nc3ncc(Cl)c(Nc4ccccc4C(N)=O)n3)c2)CC1 ; *N1CCN(c2ccc(Nc3ncc(Cl)c(Nc4ccccc4C(N)=O)n3)cc2)CC1 ; *N1CCN(c2ccc(Nc3ncc(Cl)c(Nc4ccccc4C(=O)NC)n3)cc2)CC1]
and is attached to the detectable functional element.

17. The method of claim 1, wherein the sample is selected from a cell, cell lysate, body fluid, tissue, biological sample, in vitro sample, and environmental sample.

18. The method of claim 1, wherein the kinase is expressed as a fusion with a bioluminescent reporter.

19. The method of claim 15, wherein the bioluminescent reporter is a luciferase with at least 70% sequence identity with SEQ ID NO: 4.

20. The method of claim 18, wherein the emission spectrum of the bioluminescent reporter and the excitation spectrum of the functional element overlap.

21. The method of claim 18, further comprising contacting the sample with a substrate for the bioluminescent reporter.

22. The method of claim 21, wherein the substrate is coelenterazine, coelenterazine derivative, or furimazine.

23. A system comprising:
(a) a target kinase;
(b) an active variant of KRAS; and
(c) a kinase binding agent.

24-43. (canceled)
